(19) United States
(12) Patent Application Publication
(10) Pub. No.: US 2025/0179584 A1
(43) Pub. Date: Jun. 5, 2025

(54) PRECISION THERAPY FOR THE TREATMENT OF CANCER

(71) Applicant: Novartis AG, Basel (CH)

(72) Inventor: Michele MOSCHETTA, Basel (CH)

(21) Appl. No.: 18/843,183

(22) PCT Filed: Mar. 2, 2023
(86) PCT No.: PCT/IB2023/000119

§ 371 Date: Aug. 30, 2024

Related U.S. Application Data
(60) Provisional application No. 63/315,763, filed on Mar. 2, 2022.

(51) Int. Cl. C12Q 1/6886; A61K 31/519; A61K 31/5355; A61P 35/00

(52) U.S. Cl. CPC C12Q 1/6886; A61K 31/519; A61K 31/5355; A61P 35/00; C12Q 2600/106; C12Q 2600/156

(57) ABSTRACT

The invention provides a RAF inhibitor and a MEK inhibitor for combined use in the treatment of a solid tumor, and is based on identifying certain RAS mutations, a method for identifying a patient suffering from a solid tumor for amenability to combined treatment with a RAF inhibitor and a MEK inhibitor, a corresponding method of treatment and related invention aspects or embodiments as described in detail below. The RAS mutations are in codon Q61 or a G13R mutation.

### CROSS REFERENCE

This application claims the benefit of U.S. Provisional Application No. 63/315,763, filed Mar. 2, 2022, which is incorporated herein by reference in its entirety.

### FIELD OF THE INVENTION

The invention provides a RAF inhibitor and a MEK inhibitor for combined use in the treatment of a solid tumor, and is based on identifying certain RAS mutations which can serve as prognostic markers for responsiveness to a given therapy, a method for identifying a patient suffering from a solid tumor as described herein for responsiveness to combined treatment with a RAF inhibitor (such as naporafenib or belvarafenib) and a MEK inhibitor (such as trametinib or cobimetinib), a corresponding method of treatment and related invention aspects or embodiments as described in detail below.

In particular, the present invention provides a combination of a RAF inhibitor and a MEK inhibitor for use in treating a solid tumor with a RAS codon Q61 mutation or with a G13R mutation.

In one aspect, the solid tumor to be treated is a solid tumor with a RAS codon Q61 mutation or with a G13R mutation which is not melanoma, or which is not NRAS-mutant melanoma. In another aspect, the solid tumor to be treated is a solid tumor with a RAS codon Q61 mutation or with a G13R mutation which is not rhabdomyosarcoma. In another aspect, the solid tumor to be treated is melanoma with a codon Q61 mutation in NRAS or melanoma with a G13R mutation in NRAS.

### BACKGROUND

### RAS-Mutant Cancer

RAS is the most frequently mutated gene family in cancer. Aside from NRAS, both the KRAS and to a lesser extent HRAS proto-oncogenes are frequently mutated in multiple different types of human cancers. The occurrence of RAS mutations varies widely across cancers; however, activating mutations in KRAS are found frequently in lung, colorectal and pancreatic carcinomas (Cox et al 2014; Nat. Rev. Drug Discov. 13, 828-851).

Activating mutations in KRAS, NRAS and HRAS genes alter amino acids G12, G13 and Q61 in the respective proteins in the vast majority of cases. However, the frequency with which each amino acid is altered, the specific amino acid substitution, and which RAS paralog is altered, varies significantly between cancer types. For instance, in lung adenocarcinoma, G12C variants represent >40% of the total RAS variants whereas G12C represents roughly 6% and 2% of KRAS variants found in colorectal and pancreatic cancers, respectively (Cook et al 2021). Some of this KRAS variant imbalance derives from differences in mutagenic pressures between tissue types (e.g. smoking in lung cancer). However, differences in mutagenic pressures alone do not appear to account for the majority of these allelic imbalances suggesting that different RAS variants may have unique biological properties that intersect with the etiology of different cancer types (Cook et al 2021).

Despite the fact RAS is the most frequently mutated gene family in cancer and is associated with poor outcomes, there is currently only one approved therapy targeting a RAS mutation. Sotorasib, a KRAS G12C inhibitor, demonstrated anticancer activity in patients with locally advanced or metastatic NSCLC harboring the KRAS codon G12C mutation and was granted accelerated approval by FDA in 2021. For other solid tumor types and RAS variants, there remains a significant unmet need for patients with advanced cancer who have progressed on available standard of care or for whom no standard treatment options are available.

### Melanoma

Melanoma is the most aggressive form of all skin cancers. The global incidence of melanoma is approximately 324,635 new cases per year, with 57,043 deaths (Sung et al 2020; Global Cancer Statistics 2020: GLOBOCAN Estimates of Incidence and Mortality Worldwide for 36 Cancers in 185 Countries. Available at: https://doi.org/10.3322/caac.21660 (accessed on 2 Aug. 2021)). Among cancers in patients under 40 years of age, the incidence of melanoma is the second highest only after breast cancer for women and prostate cancer for men. Most melanoma patients are diagnosed at an early stage in which surgical excision is curative with excellent survival rates (approximately 90% at 5 years). The prognosis for patients with distant metastases is, by contrast, very poor with survival rates approximately 27% at 5 years (American Cancer Society 2021; Cancer facts and figures 2021. ACS website https://www.nccn.org/professionals/physician_gls/pdf/cutaneous_melanoma.pdf. Published 2021).

The primary systemic therapy for patients with unresectable or metastatic melanoma includes anti programmed cell death protein-1 (anti-PD-1) monotherapy (i.e. nivolumab or pembrolizumab) or anti-PD-1 in combination with anti-CTLA-4 therapy (i.e. ipilimumab). In patients with melanomas harboring a BRAF V600 mutation, which occurs in ˜ 40-60% of cases (Robert et al 2019; The Lancet Oncology, Volume 20, Issue 9., KEYNOTE-006), treatment also includes targeted therapy with a BRAF inhibitor in combination with a MEK inhibitor (e.g. dabrafenib and trametinib) (NCCN Guidelines® Melanoma Version 2 2021, https://www.nccn.org/professionals/physician_gls/pdf/colon.pdf; ESMO guidelines Michielin et al 2019; Ann Oncol;30 (12): 1884-1901).

Besides BRAF mutations, the second most common mitogen activated protein kinase (MAPK) pathway aberrations in melanoma are NRAS mutations. Compared to other melanoma subtypes, melanomas with NRAS mutations are associated with a worse prognosis (Devitt et al 2011).

Selective pharmacological inhibition of NRAS remains technically challenging because its GTPase activity has so far eluded the successful design of specific small-molecule antagonists. A number of clinical trials are underway to evaluate alternative strategies which combine inhibitors of molecules downstream of NRAS, such as BRAF and MEK inhibitor combinations and the combination of PI3K pathway inhibitors with drugs that inhibit the ERK pathway. The efficacy of these various approaches is not yet established.

In fact, no targeted therapies are currently approved for NRAS-mutated melanoma, and patients with metastatic disease typically receive anti-PD-1 monotherapy or anti-PD-1 in combination with anti-CTLA4 (ipilimumab) as first-line treatment. In addition, relatlimab, a LAG-3-blocking antibody, in combination with anti-PD-1 has recently demonstrated a statistically significant PFS benefit over anti-PD-1 monotherapy in a Phase 3 trial in patients with metastatic melanoma. This regimen may become a new standard of care for this patient population if approved by health authorities. There is no established standard of care for patients with NRAS-mutant unresectable or metastatic melanoma who progress or are refractory to PD-1 inhibitors (with or without ipilimumab).

The current NCCN guidelines recommend various systemic therapies for second and subsequent lines in patients with metastatic melanoma who have failed prior anti-PD (L) 1, as monotherapy or in combination with ipilimumab, including ipilimumab monotherapy, high-dose IL-2 and cytotoxic agents such as dacarbazine, nab-paclitaxel, temozolomide or paclitaxel plus carboplatin (NCCN, loc. cit.). The ESMO guidelines acknowledge that second-line NRAS mutated metastatic melanoma treatment options are very limited. Recommendations include treatment with a MEK inhibitor (despite the known limited activity), inclusion in clinical trials, or immunotherapy re-challenge (e.g. ipilimumab if not given previously). If clinical trials or new compounds are not available, cytotoxic drugs such as dacarbazine or temozolomide may be administered (ESMO guidelines Michielin et al 2019, loc. cit.).

Ipilimumab, has been evaluated as monotherapy in a retrospective study in 162 patients with unresectable stage III/IV metastatic melanoma resistant to anti-PD (L)-1 therapy irrespective of mutation status and demonstrated a limited objective response rate (ORR) of 13%, median progression free survival (PFS) of 2.6 months and median overall survival (mOS) of 8.8 months (Da Silva et al 2021; Lancet Oncol;22 (6): 836-847)—the limited efficacy of ipilimumab monotherapy was also observed in two smaller retrospective studies. A randomized, controlled, phase 3 study evaluated dacarbazine versus the MEK inhibitor binimetinib in 402 patients with NRAS mutated melanoma, including patients untreated (˜79%) and previously treated with immunotherapy (˜21%). The ORR was 7% vs 15%, mPFS 1.7 vs 2.8 months and mOS of 10.1 vs 11 months, respectively (Dummer et al 2017; Lancet Oncol 2017; 18:435-45). Other treatments showed only limited success.

The lack of a standard of care and of approved therapies, in addition to the limited efficacy with the available therapies highlights the unmet medical need for patients with NRAS-mutant melanoma, and in particular NRAS-mutant unresectable or metastatic cutaneous melanoma.

### SUMMARY OF THE INVENTION

The present invention now provides a method of treatment for solid tumors with a mutation in RAS other than KRAS G12, e.g. other than KRAS G12C and/or other than KRAS G12D.

It has now been found that a mutation of RAS codon Q61 and/or a mutation of RAS G31R, irrespective of whether the mutation occurs in HRAS, KRAS or NRAS, is predictive of the responsiveness of a solid tumor harboring said mutation(s) to combined treatment with a RAF inhibitor and a MEK inhibitor. This finding thus provides for the treatment of any tumor, particularly a solid tumor, so long as the tumor harbors a RAS codon Q61 mutation or especially a RAS G13R mutation, and thus allows meeting a major need, offering a precise way of predicting responsiveness to treatment and treating the identified tumors. Thus the tumor to be treated does not have to be organ or tissue specific (e.g. lung cancer or pancreatic cancer or colorectal cancer or breast cancer).

In other terms, the detection and presence of a mutation of RAS codon Q61 and/or a mutation of RAS G31R, irrespective of whether the mutation occurs in HRAS, KRAS or NRAS, in a tumor or in a tumor sample obtained from a patient suffering from a tumor, and irrespective of the tissue origin of the tumor in a sample obtained from a patient suffering from a tumor (e.g. a solid tumor), allows a prognosis whether the patient suffering from such a tumor will benefit and thus respond to a combined treatment with a RAF inhibitor and a MEK inhibitor, (for example a combination of naporafenib and trametinib, or a combination of belvarafenib and cobinmetinib). The present invention thus provides a treatment of a solid tumor harboring such mutation(s).

It has now been found from a fresh and in-depth analytical review of preclinical data and past and current emerging clinical study data from clinical studies with naporafenib and other agents acting on the MPAK pathway, that a combination of a RAF inhibitor and a MEK inhibitor may be particularly beneficial for the treatment of patients suffering from pan-RAS codon Q61 or G13R mutated solid tumors.

For example, three out of four non small cell lung cancer (NSCLC) patients with tumors harboring either KRAS codon Q61 or G13R mutations responded to treatment with a naporafenib and trametinib combination, versus 1 out of 43 NSCLC patients with tumors harboring KRAS mutations other than either codon Q61 or G13R mutations. In addition, as shown from clinical data from the LXH254 first in human study (CLXH254X2101), patients with solid tumors (salivary gland tumor and NSCLC) harboring pan-RAS codon Q61 or G13R mutations were found to be exceptional responders to LXH254 single agent therapy. Moreover, non-clinical models have shown consistent activity of LXH254 as a single agent or in combination with trametinib in pan-RAS codon Q61/G13R mutant models across tumor types (NSCLC, melanoma, PDAC, rhabdomyosarcomas), independently of the particular mutated RAS gene (pan-RAS) and the tissue origin of the tumor (tissue agnostic). Overall, this totality of data suggests that pan-RAS codon Q61/G13R mutations may represent predictive biomarkers of response to a combination of a RAF inhibitor and a MEK inhibitor.

This data, along with pre-clinical evidence, as well as clinical data with belvarafenib, another RAF inhibitor, makes it plausible that codon Q61/G13R (pan-) RAS mutations represent a predictive biomarker of response to a combination of a RAF inhibitor (such as naporafenib or belvarafenib) and a MEK inhibitor (such as trametinib or cobimetinib) combination.

In addition, in a molecular dynamic simulation analysis performed at Novartis, G13R was found to overlap with Q61 and to behave dynamically similarly and to presumably adopt a similar conformation as Q61. Thus, mutations RAS Q61 and G13R in a RAS gene, whether it is a RAS gene selected from HRAS, KRAS, NRAS genes, and combinations thereof are appropriate biomarkers which allow the identification of tumor subtypes that are strongly MAPK-dependent and likely to benefit from the combination therapy. Once the MAPK pathway is activated, homo-or heterodimers of RAF activate MEK1 and MEK2 that are the main downstream signaling nodes. Given RAF and MEK are vertically integrated in the same pathway, a combined inhibition of these two nodes may provide more effective inhibition of the pathway and delay development of resistance.

It is therefore the basis of the present invention that by using RAS codon Q61 or especially RAS G13R mutations as predictive biomarkers for a solid tumor that can be treated by a RAF inhibitor and MEK inhibitor combination treatment it is possible to select and treat patients with tumors that harbor such mutation(s). G13R mutations have been found here to be an important predictive biomarker.

The present invention thus provides the following embodiments:

Embodiment 1. A method of selectively treating a patient suffering from a solid tumor, comprising selectively administering a therapeutically effective amount of a pharmaceutical combination of a RAF inhibitor and a MEK inhibitor to the patient on the basis of the tumor having a RAS codon Q61 mutation or a G13R mutation.

Embodiment 2. A method of selectively treating a patient suffering from a solid tumor having a RAS codon Q61 mutation or a G13R mutation with a pharmaceutical combination of a RAF inhibitor and a MEK inhibitor, comprising:
a) selecting the patient for treatment with a pharmaceutical combination of a RAF inhibitor and a MEK inhibitor on the basis of the tumor having a RAS codon Q61 mutation or a G13R mutation; and
b) thereafter, administering a therapeutically effective amount of a pharmaceutical combination of a RAF inhibitor and a MEK inhibitor to the patient.

Embodiment 3. A method of selectively treating a patient suffering from a solid tumor having a RAS codon Q61 mutation or a G13R mutation, comprising:
a) assaying a biological sample or a tumor sample from the patient for the presence of a RAS codon Q61 mutation or a G13R mutation in the solid tumor; and
b) thereafter, selectively administering to the patient either:
i. a therapeutically effective amount of a pharmaceutical combination of a RAF inhibitor and a MEK inhibitor when a RAS codon Q61 mutation or a G13R mutation is present; or
ii. a therapeutically effective amount of a Drug other than a pharmaceutical combination of a RAF inhibitor and a MEK inhibitor on the basis of a RAS codon Q61 mutation or a G13R mutation not being present or not detected in the biological or tumor sample from the patient.

Embodiment 4. A method of selectively treating a patient suffering from a solid tumor having a RAS codon Q61 mutation or a G13R mutation with a pharmaceutical combination of a RAF inhibitor and a MEK inhibitor, comprising:
a) assaying a biological sample or a tumor sample from the patient for the presence of a RAS codon Q61 mutation or a G13R mutation;
b) thereafter, selecting the patient for treatment with a pharmaceutical combination of a RAF inhibitor and a MEK inhibitor on the basis of the biological or tumor sample from the patient having a RAS codon Q61 mutation or a G13R mutation; and
c) thereafter, administering a therapeutically effective amount of a pharmaceutical combination of a RAF inhibitor and a MEK inhibitor to the patient.

Embodiment 5. The method according to any one of embodiments 3 or 4, wherein the biological sample is selected from the group consisting of synovial fluid, blood, serum, feces, plasma, urine, tear, saliva, cerebrospinal fluid, a leukocyte sample and a tumor sample (also referred to as a tumor biopsy).

Embodiment 6. The method according to any one of embodiments 3-5, wherein the step of assaying comprises a technique selected from the group consisting of Northern blot analysis, polymerase chain reaction (PCR), reverse transcription-polymerase chain reaction (RT-PCR), TaqMan-based assays, direct sequencing, dynamic allele-specific hybridization, high-density oligonucleotide SNP arrays, restriction fragment length polymorphism (RFLP) assays, primer extension assays, oligonucleotide ligase assays, analysis of single strand conformation polymorphism, temperature gradient gel electrophoresis (TGGE), denaturing high performance liquid chromatography, high-resolution melting analysis, DNA mismatch-binding protein assays, SNPLex®, capillary electrophoresis, Southern Blot, immunoassays, immunohistochemistry, ELISA, flow cytometry, Western blot, HPLC, and mass spectrometry.

Embodiment 7. A pharmaceutical combination of a RAF inhibitor and a MEK inhibitor for use in treating a patient suffering from a solid tumor having a RAS codon Q61 mutation or a G13R mutation, characterized in that a pharmaceutical combination of a RAF inhibitor and a MEK inhibitor is administered to the patient on the basis of said patient having a solid tumor having a RAS codon Q61 mutation or a G13R mutation.

Embodiment 8. A pharmaceutical combination of a RAF inhibitor and a MEK inhibitor for use in the treatment suffering from a solid tumor having a RAS codon Q61 mutation or a G13R mutation in a patient, comprising
a) assaying a sample from the patient,
b) determining if the sample shows the presence of a solid tumor having a RAS codon Q61 mutation or a G13R mutation, and
c) if the RAS codon Q61 mutation or a G13R mutation is present, a therapeutically effective amount of the pharmaceutical combination of a RAF inhibitor and a MEK inhibitor is to be administered to the patient.

Embodiment 9. A pharmaceutical combination of a RAF inhibitor and a MEK inhibitor for use in treating a patient suffering from a solid tumor having a RAS codon Q61 mutation or a G13R mutation, characterized in that:
a) the patient is selected for treatment with the pharmaceutical combination of a RAF inhibitor and a MEK inhibitor on the basis of the patient suffers from a solid tumor with a RAS codon Q61 mutation or a G13R mutation; and
b) thereafter, a therapeutically effective amount of a pharmaceutical combination of a RAF inhibitor and a MEK inhibitor is administered to the patient.

Embodiment 10. A pharmaceutical combination of a RAF inhibitor and a MEK inhibitor for use in treating a patient suffering from a solid tumor having a RAS codon Q61 mutation or a G13R mutation, characterized in that:
a) a biological sample from the patient is assayed for having a RAS codon Q61 mutation or a G13R mutation; and
b) a therapeutically effective amount of a pharmaceutical combination of a RAF inhibitor and a MEK inhibitor is selectively administered to the patient on the basis of the biological sample from the patient having a RAS codon Q61 mutation or a G13R mutation.

Embodiment 11. A pharmaceutical combination of a RAF inhibitor and a MEK inhibitor for use in treating a patient suffering from a solid tumor having a RAS codon Q61 mutation or a G13R mutation, characterized in that:
a) a biological sample or a tumor sample from the patient is assayed for the presence of a RAS codon Q61 mutation or a G13R mutation;
b) the patient is selected for treatment with a pharmaceutical combination of a RAF inhibitor and a MEK inhibitor on the basis of the biological sample from the patient having a RAS codon Q61 mutation or a G13R mutation; and
c) a therapeutically effective amount of pharmaceutical combination of a RAF inhibitor and a MEK inhibitor is to be selectively administered to the patient.

Embodiment 12. A method of predicting the likelihood that a patient suffering from a solid tumor with a RAS codon Q61 mutation or a G13R mutation will respond to treatment with a pharmaceutical combination of a RAF inhibitor and a MEK inhibitor, comprising assaying a biological sample or a tumor sample from the patient for the presence or absence of having a RAS codon Q61 mutation or a G13R mutation, wherein:
a) the presence of a RAS codon Q61 mutation or a G13R mutation is indicative of an increased likelihood that the patient will respond to treatment with a pharmaceutical combination of a RAF inhibitor and a MEK inhibitor; and
b) the absence of having a RAS codon Q61 mutation or a G13R mutation is indicative of a decreased likelihood that the patient will respond to treatment with a pharmaceutical combination of a RAF inhibitor and a MEK inhibitor.

Embodiment 13. The method according to embodiment 12, further comprising the step of obtaining the biological sample from the patient, wherein the step of obtaining is performed prior to the step of detecting.

Embodiment 14. The method according to any one of embodiments 12-13, wherein the biological sample is selected from the group consisting of synovial fluid, blood, serum, feces, plasma, urine, tear, saliva, cerebrospinal fluid, a leukocyte sample a tissue sample, and a tumor sample.

Embodiment 15. The method according to any one of embodiments 12-14, wherein the step of assaying comprises a technique selected from the group consisting of Northern blot analysis, polymerase chain reaction (PCR), reverse transcription-polymerase chain reaction (RT-PCR), TaqMan-based assays, direct sequencing, dynamic allele-specific hybridization, high-density oligonucleotide SNP arrays, restriction fragment length polymorphism (RFLP) assays, primer extension assays, oligonucleotide ligase assays, analysis of single strand conformation polymorphism, temperature gradient gel electrophoresis (TGGE), denaturing high performance liquid chromatography, high-resolution melting analysis, DNA mismatch-binding protein assays, SNPLex®, capillary electrophoresis, Southern Blot, immunoassays, immunohistochemistry, ELISA, flow cytometry, Western blot, HPLC, and mass spectrometry.

Embodiment 16. A method for producing a transmittable form of information for predicting the responsiveness of a patient suffering from a solid tumor to treatment with a pharmaceutical combination of a RAF inhibitor and a MEK inhibitor, comprising:
a) determining an increased likelihood of the patient responding to treatment with a pharmaceutical combination of a RAF inhibitor and a MEK inhibitor based on the presence of a RAS codon Q61 mutation or a G13R mutation in the tumor; and
b) recording the result of the determining step on a tangible or intangible media form for use in transmission.

Embodiment 17. A kit for use in predicting the likelihood that a patient suffering from a solid tumor will respond to treatment with a pharmaceutical combination of a RAF inhibitor and a MEK inhibitor comprising,
a) at least one probe capable of detecting the presence of having a RAS codon Q61 mutation or a G13R mutation; and
b) instructions for using the probe to assay a biological sample from the patient for the presence of a RAS codon Q61 mutation or a G13R mutation, wherein the presence of a RAS codon Q61 mutation or a G13R mutation is indicative of an increased likelihood that the patient will respond to treatment with a pharmaceutical combination of a RAF inhibitor and a MEK inhibitor and the absence of the having a RAS codon Q61 mutation or a G13R mutation is indicative of a decreased likelihood that the patient will respond to treatment with a pharmaceutical combination of a RAF inhibitor and a MEK inhibitor.

Embodiment 18. A kit for use in treating a patient suffering from a solid tumor comprising,
a) a therapeutically effective amount of a pharmaceutical combination of a RAF inhibitor and a MEK inhibitor;
b) at least one probe capable of detecting the presence of a RAS codon Q61 mutation or a G13R mutation in the solid tumor;
c) instructions for using the probe to assay a biological sample or a tumor sample from the patient for the presence of a RAS codon Q61 mutation or a G13R mutation,
d) instructions for administering the pharmaceutical combination of a RAF inhibitor and a MEK inhibitor to the patient if the biological sample or tumor sample from the patient indicates the presence of a RAS codon Q61 mutation or a G13R mutation; and
e) optionally, means for administering the a pharmaceutical combination of a RAF inhibitor and a MEK inhibitor to the patient.

Embodiment 19. The kit according to any one of embodiments 17 or 18, wherein the probe is an oligonucleotide that specifically hybridizes to a region of a nucleic acid coding for a RAS codon Q61 mutation or a G13R mutation, an antibody that detects a polypeptide product of the RAS codon Q61 mutation or a G13R mutation, or an oligonucleotide that specifically hybridizes to a region of a nucleic acid coding for an equivalent genetic marker of the RAS codon Q61 mutation or a G13R mutation.

In the embodiments above, the solid tumor with a RAS codon Q61 mutation or with a G13R mutation may be selected from:
a solid tumor which is not melanoma, or which is not NRAS-mutant melanoma;
a solid tumor which is not rhabdomyosarcoma;
a solid tumor which is melanoma with a codon Q61 mutation in NRAS or melanoma with a G13R mutation in NRAS;
a solid tumor which is selected from non-small cell lung cancer, pancreatic cancer, thyroid cancer and colorectal cancer.

### BRIEF DESCRIPTION OF THE DRAWINGS

FIG. 1 is a graphic representation of KRAS G13R protein dynamic overlap with KRAS Q61R but not G13D.

FIG. 2 is a diagram showing an overview of the Study Design for a study concerning mutated melanoma and solid tumor patients.

FIG. 3 shows the anti-tumor activity of naporafenib and trametinib across ten patients derived NRASmut melanoma tumor xenograft models in mice.

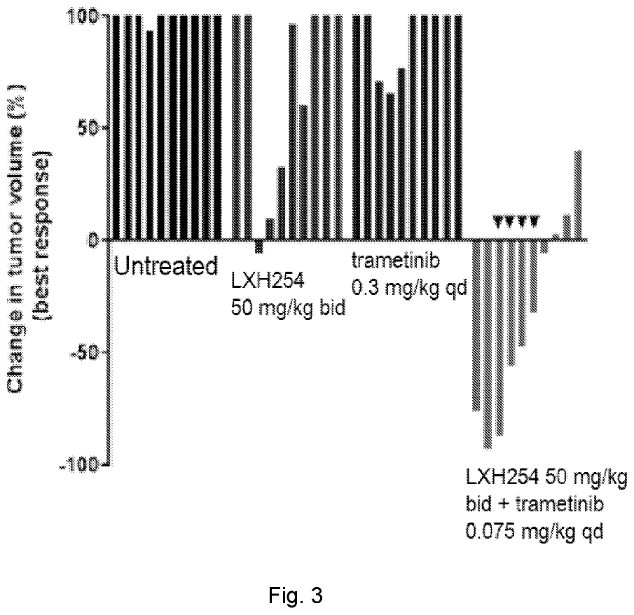

### DETAILED DESCRIPTION OF THE INVENTION

In one embodiment, the invention provides an agent or a probe capable of identifying a RAS codon Q61 or especially RAS G13R mutation for (or in particular for use in) diagnosing a solid tumor, in particular other than melanoma, yet more preferably other than melanoma or other than rhabdomyosarcoma, for amenability to a combination treatment with a Raf inhibitor and a MEK inhibitor, said diagnosing comprising applying the agent to assay a sample of said solid tumor whether it harbors a RAS codon Q61 or RAS G13R mutation as biomarker for said amenability.

In one embodiment, the present invention provides a method of identifying a patient suffering from a solid tumor, in particular a solid tumor other than melanoma, yet more preferably a solid tumor other than melanoma or a solid tumor other than rhabdomyosarcoma, for an increased likelihood of response to a combined treatment with a Raf inhibitor and a MEK inhibitor, the method comprising:
(a) assaying a tumor sample obtained from the patient for the presence of a RAS codon Q61 or RAS G13R mutation with an agent according to the preceding paragraph; and
(b) selecting a patient whose tumor sample was positively assayed under (a) for the presence of said RAS codon Q61 or RAS G13R mutation as candidate for said combination treatment.

In another embodiment, the present invention provides a method for selecting a treatment for a patient suffering from a solid tumor, the method including screening a tumor sample from the patient for a RAS codon Q61 or (especially) RAS G13R mutation, wherein the presence of said mutation(s) in the tumor sample identifies the patient as one who may be expected to benefit from a treatment including a Raf inhibitor and a MEK inhibitor.

In another embodiment, the present invention provides a method of treating a patient suffering from a solid tumor, the method comprising
(a) examining a tumor sample from the patient suffering from the tumor whether it harbors a RAS codon Q61/or RAS G13R mutation, and,
(b) where the tumor sample has been determined to harbor said mutation, administering in a combined treatment of therapeutically effective amounts a Raf inhibitor and a MEK inhibitor.

Another embodiment of the invention provides a method of treatment of a patient suffering from a solid tumor, in particular other than melanoma, in need of such treatment, comprising administering to said patient, where the solid tumor was identified to harbor a RAS codon Q61 or RAS G13R mutation, a combination of, in an effective amount, a RAF inhibitor and a MEK inhibitor.

Another embodiment of the invention provides a kit, comprising an agent for identifying a RAS codon Q61 r RAS G13R mutation and reagents for a method according to any one of the preceding paragraphs.

A further embodiment of the invention provides a Raf inhibitor and a MEK inhibitor for combined use in the treatment of a solid tumor, in particular other than melanoma and/or rhabdomyosarcoma harboring a NRAS codon Q61 mutation, in a patient where the tumor has been positively tested and identified to harbor a RAS codon Q61 or RAS G13R mutation.

A further embodiment of the invention provides the Raf inhibitor and the MEK inhibitor for combined use according to the preceding paragraph, where the Raf inhibitor is selected from the group consisting of encorafenib; sorafenib, vemurafenib, dabrafenib, GDC-0879, PLX-4720, PLX8394, belvarafenib, CCT3833/BAL3833, LY3009120, LSN3074753, lifirafenib, BGB659, RO5126766, AZ-628, MLN2480, BeiGene-283, RXDX-105, BAL3833, INU152, regorafenib, tovorafenib, Dayl01, TAK580, MLN 2480, TAK632 and especially naporafenib (LXH254; Type II); or a pharmaceutically acceptable salt, hydrate or solvate thereof, respectively.

A further embodiment of the invention provides the Raf inhibitor and the MEK inhibitor for combined use according to any one of the preceding two paragraphs, where the Raf inhibitor is naporafenib, or a pharmaceutically acceptable salt, hydrate or solvate thereof.

A further embodiment of the invention provides the Raf inhibitor and the MEK inhibitor for combined use according to any one of the preceding paragraphs, wherein the MEK inhibitor is selected from the group consisting of binimetinib, trametinib, selumetinib, cobimetinib, CI-1040, U0126-EtOH, PD198306, PD98059, BIX 02189, TAK-733, Honoliol, AZD8330, PD318088, BIX 02188, pimasertib, mirdametinib, refametinib, BI-847325, GDC0623, G-573, trametiglue, RO5126766 and pimisertib, or a pharmaceutically acceptable salt, hydrate or solvate thereof.

Preferred MEK inhibitors include trametinib, binimetinib and cobmimetinib.

A further invention embodiment provides the Raf inhibitor and MEK inhibitor for combined use according to any one of the preceding four paragraphs, wherein the MEK inhibitor is trametinib, or a pharmaceutically acceptable salt, hydrate or solvate thereof.

A further embodiment of the invention provides the Raf inhibitor and MEK inhibitor for combined use according to any one of the preceding paragraphs, where the solid tumor has been positively tested to harbor a RAS G13R mutation.

A further embodiment of the invention provides the combined use of a Raf inhibitor and a MEK inhibitor, especially as defined in more detail herein, especially naporafenib and trametinib, or a pharmaceutically acceptable salt, hydrate or solvate thereof, respectively, in the treatment of a solid tumor in a patient, where the tumor has been positively tested to harbor a RAS codon Q61or RAS G13R mutation.

A further embodiment of the invention provides the use of a Raf inhibitor and a MEK inhibitor, especially as defined in more detail herein, especially naporafenib and trametinib, or a pharmaceutically acceptable salt, hydrate or solvate thereof, respectively, in the preparation of (especially combination) medicaments, e.g. kits for combined use or fixed combination formulations comprising both active ingredients, for combination treatment of a solid tumor in a patient, where the solid tumor has been determined to harbor a RAS codon Q61or RAS G13R mutation.

Another embodiment of the invention provides a composition comprising a Raf inhibitor and a MEK inhibitor, especially as defined in more detail herein, especially naporafenib and trametinib, or a pharmaceutically acceptable salt, hydrate or solvate thereof, respectively, and a pharmaceutically acceptable carrier in the form of separate formulations or a kit or a fixed formulation for use in the treatment of a solid tumor selected from rhabdomyosarcoma identified to harbor a RAS G13R mutation, or a different solid tumor, especially other than melanoma, identified to harbor a RAS codon Q61 or RAS G13R mutation; or a corresponding method of treatment comprising administering the Raf inhibitor and MEK inhibitor mentioned in a combined therapeutically active amount to a patient suffering from said rhabdomyosarcoma or other solid tumor in need of such treatment.

The more particular definitions used herein, especially below, may be used to replace one, more or all broader terms, features or expressions of invention embodiments herein, thus defining further invention embodiments.

Amenability to the combination/combined treatment indicates that a positive effect on a tumor disease can be expected—for definition of such disease see below. In other terms, it means that a patient may benefit from such treatment, allowing an improved prognosis.

RAS herein refers to HRAS, NRAS and KRAS.

### RAF Inhibitors

Raf inhibitors as mentioned herein includes any RAF inhibitor, also e.g. specific (e.g. ARAF, BRAF or CRAF specific) inhibitors, but mainly inhibitors for two or all three of ARAF, BRAF and CRAF. Preferred classes of RAF inhibitors include Type I inhibitors, Type 1.5 inhibitors (including so-called paradox breakers) and Type II inhibitors.

Paradox breakers are a sub-species of type 1.5 inhibitors. In cells type 1.5 inhibitors such as encorafenib, vemurafenib and dabrafenib, activate signaling by wild type, but not BRAFV600E mutant BRAF. This surprising activation by an inhibitor was termed paradoxical activation. The “paradox breakers” are a type of 1.5 inhibitors that did not this induce paradoxical activation.

Preferred RAF inhibitors are so-called Type II inhibitors.

A preferred Raf inhibiting compound is selected from the group consisting of (where some known types are mentioned in parenthesis):
sorafenib (BAY43-9006), belvarafenib (HM95573), CCT3833/BAL3833, LY3009120, LSN3074753, lifirafenib (BGB-283), BGB659, RO5126766, AZ-628, MLN2480, BeiGene-283 (BGB283), RXDX-105, BAL3833, INU152, regorafenib, tovorafenib, Dayl01, TAK580, MLN 2480, TAK632 and especially naporafenib (LXH254), or a pharmaceutically acceptable salt, hydrate or solvate thereof, respectively.

More preferably, the RAF inhibitor compound may be selected from sorafenib (BAY43-9006; Type II), belvarafenib (HM95573; Type II; preferred), CCT3833/BAL3833, LSN3074753, lifirafenib (BGB-283; Type II), BGB659, RO5126766, AZ-628, MLN2480 (Type II), BeiGene-283 (BGB283), RXDX-105, BAL3833, INU152, regorafenib (Type II), tovorafenib, Day 101, TAK580, MLN 2480, TAK632 and especially naporafenib (LXH254; Type II); or a pharmaceutically acceptable salt, hydrate or solvate thereof, respectively.

Preferred RAF inhibitors include:
Naporafenib (LXH254)
Naporafenib has the formula I

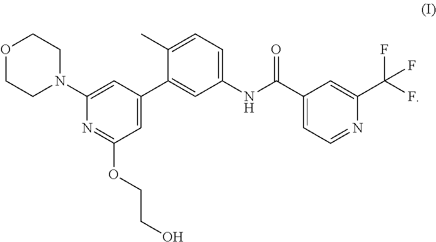 [SMILES: Cc1ccc(NC(=O)c2ccnc(C(F)(F)F)c2)cc1-c1cc(OCCO)nc(N2CCOCC2)c1]

This compound is also known by the name N-(3-(2-(2-hydroxyethoxy)-6-morpholino-pyridin-4-yl)-4-methylphenyl)-2-(trifluoromethyl) isonicotinamide or N-{3-[2-(2-Hydro-xyethoxy)-6-(morpholin-4-yl) pyridin-4-yl]-4-methylphenyl}-2-(trifluoromethyl)pyridine-4-carboxamide. Wherever naporafenib is mentioned herein, this refers to the free compound itself but is in addition further defined here to include a pharmaceutically acceptable salt, hydrate and/or solvate thereof. Especially preferred is naporafenib in free form or as a monohydrate such as Monohydrate Form HA characterized by having an X-ray powder diffraction pattern with at least one, two, three, four or five peaks having an angle of refraction 2 theta (θ) values selected from 7.3, 10.7, 16.3, 16.7, 17.4, 23.0, 24.3, 25.3, 28.3, 32.0 when measured using CuKa radiation, wherein said values are plus or minus 0.2° 2θ. Monohydrate Form HA of naporafenib is further described in and can be prepared as described in WO/2020/230028.

Naporafenib is an adenosine triphosphate (ATP)-competitive inhibitor mainly of the BRAF and CRAF protein kinases. Because naporafenib can inhibit both monomeric and dimerized B- and-CRAF, it induces minimal paradoxical activation and is effective at blocking mutant RAS-driven signaling and cell proliferation. Naporafenib has demonstrated efficacy in a wide range of MAPK pathway-driven human cancer cell lines and in vivo tumor xenografts including models harboring activating lesions in the KRAS, NRAS, and BRAF oncogenes.

The compound of formula (I) and its pharmaceutically acceptable salts are described in WO2014/151616, which is hereby incorporated by reference in its entirety, and methods of its preparation have been described, for example, in Example 1156 therein. Naporafenib may also be used as a polymorphic form thereof, e.g. as described in WO/2020/230028.

In cell-based assays, the compound of formula (I) has demonstrated anti-proliferative activity in cell lines that contain a variety of mutations that activate MAPK signaling. In vivo, treatment with the compound of formula (I) generated tumor regression in several KRAS-mutant models. Collectively, the in vitro and in vivo MAPK-pathway suppression and anti-proliferative activity observed for the compound of formula (I) at well-tolerated doses make it plausible that the compound of formula (I) may have anti-tumor activity in patients with tumors harboring activating lesions in the MAPK pathway. Moreover, the compound of formula (I) is a Type 2 ATP-competitive inhibitor of both B-RAF and C-RAF that keeps the kinase pocket in an inactive conformation, thereby blocking mutant RAS-driven signaling and cell proliferation. The compound of formula (I) has exhibited efficacy in numerous MAPK-driven human cancer cell lines and in xenograft tumors representing model tumors harboring human lesions in KRAS, NRAS and BRAF oncogenes (see e.g. WO/2018/203219, which is hereby incorporated in its entirety).

Further evidence for the utility of the present invention may be derived from the clinical data obtained from LXH254, e.g.
CLXH254X2101: a first-in-human study of naporafenib as single agent in patients with advanced solid tumors harboring MAPK pathway alterations and in combination with the checkpoint inhibitor spartalizumab (PDR001) in patients with advanced or metastatic KRAS mutant non-Small Cell Lung Cancer or NRAS mutant melanoma.
CLXH254X2102: a Phase Ib, open-label, multicenter study of naporafenib in combination with LTT462 in adult patients with advanced or metastatic KRAS or BRAF mutant non-Small Cell Lung Cancer, or with trametinib in adult patients with advanced or metastatic KRAS or BRAF mutant non-Small Cell Lung Cancer or NRAS mutant melanoma, or with ribociclib in adult patients with advanced or metastatic NRAS mutant melanoma.
CLXH254C12201: a randomized, open-label, multi-arm, two-part, phase II study to assess the efficacy and safety of multiple naporafenib combinations in patients with previously treated unresectable or metastatic BRAF V600 or NRAS mutant melanoma.

### Belvarafenib

Belvarafenib (HM95573, GDC5573, RG6185) has the formula

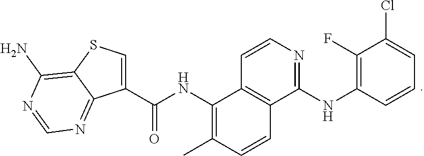 [SMILES: Cc1ccc2c(Nc3cccc(Cl)c3F)nccc2c1NC(=O)c1csc2c(N)ncnc12]

Belvarafenib has the name 4-amino-N-(1-((3-chloro-2-fluorophenyl)amino)-6-methylisoquinolin-5-yl) thieno[3,2-d]pyrimidine-7-carboxamide and is a potent pan RAF inhibitor with antineoplastic activity, especially for BRAF, BRAF V600E and CRAF. Its synthesis is described in WO2013/100632 A1. The compound shows high selectivity toward BRAF mutant and CRAF kinases. A phase Ib trial of belvarafenib in combination with cobimetinib in patients with advanced solid tumors is being conducted: https://ascopubs.org/doi/abs/10.1200/JCO.2021.39.15_suppl.3007

### MEK Inhibitors

The term “MEK inhibitor” is defined herein to refer to a compound which targets, decreases or inhibits at least one activity of MAP/ERK kinases 1 and 2 (MEK1/2). MEK stands for “mitogen-activated protein kinase”.

MEK inhibitors as mentioned herein includes inhibitors of both MEK and MEK/RAF heterodimer complexes.

Preferred MEK inhibitory compounds are selected from the group consisting of: Arry-162=MEK162 (binimetinib), AZD6244 (selumetinib), XL518=GDC0973 (cobimetinib), CI-1040=PD184352, U0126-EtOH, PD198306, PD98059, BIX 02189, TAK-733, Honoliol, AZD8330, PD318088, BIX 02188, AS703026=MSC1936369B (pimasertib), SL327, PD-325901=PD0325901 (mirdametinib), TAK-733, BAY86-9766 (refametinib), BI-847325, GDC0623, G-573, RAF/MEK heterodimer breaker), trametiglue (RAF/MEK complex inhibitor), CH4987655=RO 4987655, WX-554, HL-085, CH5126766=RO5126766 (RAF/MEK complex inhibitor) (preferred), and especially trametinib (GSK1120212; advanced RAF inhibitor; marketed under the tradename Mekinist®.

Preferred MEK inhibitors include trametinib, binimetinib and cobmimetinib.

Preferably, the MEK inhibitor is trametinib, or a pharmaceutically acceptable salt or solvate thereof. In some embodiments, trametinib is in the form of a sodium salt. Suitably, trametinib is in the form of a solvate selected from: hydrate, acetic acid, ethanol, nitromethane, chlorobenzene, 1-pentancol, isopropyl alcohol, ethylene glycol and 3-methyl-1-butanol. In some preferred embodiments, trametinib is in the form of a dimethyl sulfoxide solvate. These solvates and salt forms can be prepared by one of skill in the art from the description in WO 2005/121142.

### Trametinib has the Formula II

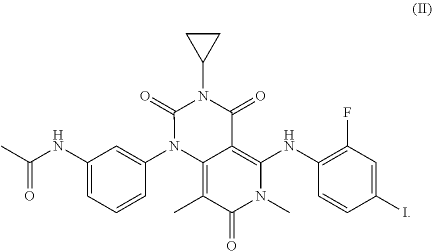 [SMILES: CC(=O)Nc1cccc(-n2c(=O)n(C3CC3)c(=O)c3c(Nc4ccc(I)cc4F)n(C)c(=O)c(C)c32)c1]

This compound is also known by its name which is (N-(3-{3-cyclopropyl-5-[(2-fluoro-4-iodophenyl)amino]-6,8-dimethyl-2,4,7-trioxo-3,4,6,7-tetrahydropyrido[4,3-d]pyrimidin-1 (2H)-yl}phenyl) acetamide), and has also been referred to as JPT-74057 or GSK1120212 or TMT212.

Trametinib is disclosed in Example 4-1 in PCT Publication No. WO 2005/121142, which is hereby incorporated by reference in its entirety.

Wherever “trametinib” is made reference to in the present invention disclosure, this refers to the free form itself but is also defined here to include a pharmaceutically acceptable salt, hydrate and/or solvate thereof, particularly the dimethyl sulfoxide solvate.

RO5126766 has the formula III,

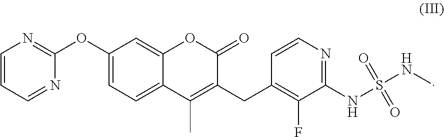

The compound has the name N-(3-fluoro-4-((4-methyl-2-oxo-7-(7-(2-pyrimidinyloxy)-2H-1-benzopyran-3-yl)methyl-2-pyridinyl)-N′-methyl-sulfamide. RO5126766 is a dual RAF/MEK inhibitor with IC50 of 8.2 nM, 19 nM, 56 nM and 160 nM for BRAF V600E, BRAF, CRAF and MEK1, respectively.

“Furthermore” or “further” indicates that features following any of these words are less preferred than features without this attribute.

As used herein, the terms “a” and “an” and “the” and similar references in the context of describing the invention are to be construed to cover both the singular and the plural, unless otherwise indicated herein or clearly contradicted by context. Where the plural form is used for compounds, salts, and the like, this is taken to mean also a single compound, salt, or the like.

The term “or” is used herein to mean, and is used interchangeably with, the term “and/or”, unless context clearly indicates otherwise, e.g. reference to a compound or a pharmaceutically acceptable salt thereof provides the free compound, a pharmaceutically acceptable salt, or a mixture of free compound and one or further more salts thereof.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values. When describing a dosage herein as “about” a specified amount, the actual dosage can vary by up to 10% from the stated amount: this usage of “about” recognizes that the precise amount in a given dosage form may differ slightly from an intended amount for various reasons without materially affecting the in vivo effect of the administered compound.

It will be understood that when a numerical value is given, without the term “about” preceding it, there will be a degree of variation associated with that given value, as is commonly accepted in the art; for example, the variation may be ±10%, e.g. ±5%, in particular±1% of the given numerical value, respectively.

The terms “comprising” and “including” or “with” are used herein in their open-ended and non-limiting sense unless otherwise noted, while “consisting of”′ limits the compound or components to those specifically mentioned. A pharmaceutical combination consisting of naporafenib, or a pharmaceutically acceptable salt, hydrate or solvate thereof, and (b) trametinib, or a pharmaceutically acceptable salt, hydrate or solvate thereof refers to a combination therapy where these two components are the only pharmaceutically active ingredients; with or without one or more pharmaceutically acceptable carrier materials.

By “a combination” or “a pharmaceutical combination” (these terms being synonymous if not suggested otherwise) or “in combination with” or “combination therapy” it is not only intended to imply that the therapy or the therapeutic agents must be physically mixed or administered at the same time and/or formulated for delivery together, although these methods of delivery are within the scope described herein. A therapeutic agent (which term herein primarily refers to a RAF inhibitor and/or a MEK inhibitor, especially naporafenib and/or trametinib) in these combinations can furthermore be administered concurrently with, prior to, or subsequent to, one or more other additional therapies or additional therapeutic agents, or especially without such additional therapies or additional therapeutic agents (active ingredients). The therapeutic agents can be administered in any order. In general, each agent will be administered at a dose and/or on a time schedule determined for that agent. It will further be appreciated that the additional therapeutic agent utilized in this combination may be administered together in a single composition and/or administered separately in different compositions, especially where these time intervals allow that the combination partners show a cooperative, e.g., synergistic, effect, especially based on Bliss data or other data confirming synergy. In general, it is expected that additional therapeutic agents utilized in combination be utilized at levels that do not exceed the levels at which they are utilized individually. In some embodiments, the levels for each involved therapeutic agent=drug=active pharmaceutical ingredient (API) utilized in combination will be lower than those utilized as single-agent therapeutics.

The term “synergistic effect” as used herein, refers to the action of a RAF inhibitor, especially naporafenib, or a pharmaceutically acceptable salt, hydrate and/or solvate thereof, and a MEK inhibitor, especially trametinib, a pharmaceutically acceptable salt, hydrate and/or solvate thereof, to produce an effect, for example, slowing the symptomatic progression of cancer or symptoms thereof, which is greater than the simple addition of the effects of each drug administered by themselves or required a lower dosage of any one or both therapeutic agents.

The term “combination therapy” preferably refers to the administration of two or more of the therapeutic agents to treat a tumor condition or disorder described in the present invention. Such administration encompasses co-administration of these therapeutic agents in a substantially simultaneous manner, such as in a single formulation having a fixed ratio of active ingredients, or in separate formulations (e.g., capsules, tablets and/or further intravenous formulations) for each active ingredient. In addition or alternatively, such administration also encompasses use of each type of therapeutic agent in a sequential or separate manner, either at approximately the same time or at different times. Regardless of whether the active ingredients are administered as a single formulation or in separate formulations, the drugs are administered to the same patient as part of the same course of therapy. In any case, the treatment regimen will provide beneficial effects in treating the tumor conditions or disorders described herein.

By simultaneous therapeutic use, within the meaning of the present invention is meant in particular an administration of at least two active ingredients by the same route and at the same time or at substantially the same time.

By separate use, within the meaning of the present invention is meant in particular an administration of at least two active ingredients at the same time or at substantially the same time by different routes or formulations.

By sequential therapeutic use is meant in particular administration of at least two active ingredients at different times, the administration route or formulation being identical or different. More particularly by an administration method is meant according to which the whole administration of one of the active ingredients is carried out before administration of the other (or further others) commences.

The synonymous terms “fixed combination”, “fixed dose” and “single formulation” as used herein preferably refers to one dosage form formulated to deliver an amount, which is jointly therapeutically effective for the treatment of cancer, of both therapeutic agents to a patient. The single vehicle is designed to deliver an amount of each of the agents along with any pharmaceutically acceptable carriers (excipients). In some embodiments, the vehicle is a tablet, capsule, pill, a sachet or a patch. In further embodiments, the vehicle is a solution or a suspension.

The term combination also or alternatively includes a “non-fixed combination” or “kit of parts” which means that the therapeutic agents of the combination of the invention are both administered to a patient as separate entities either simultaneously, concurrently or sequentially with no specific time limits, especially so that they are jointly therapeutically active, wherein such administration provides therapeutically effective levels of the two compounds in the body of a patient in need thereof. The latter further also applies to cocktail therapy, e.g., the administration of three or more active ingredients.

The term “pharmaceutically acceptable” as used herein refers to those compounds, materials, excipients, compositions and/or dosage forms, which are, within the scope of sound medical judgment, suitable for contact with the tissues of a patient, e.g., an adult patient or a human patient, without excessive toxicity, irritation, allergic response and other problems or complications commensurate with a reasonable and tolerable benefit/risk ratio.

As used herein, the term “pharmaceutically acceptable excipient” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, diluents, disintegration agents, glidants, lubricants, sweetening agents, flavoring agents, dyes, and the like and combinations thereof, as would be known to those skilled in the art. Except insofar as any conventional carrier is incompatible with the active ingredient, its use in the therapeutic or pharmaceutical compositions is comprised.

The term “pharmaceutical composition” (=pharmaceutical formulation) is defined herein to refer to a mixture or solution containing at least one therapeutic agent to be administered to a patient, e.g., a mammal or human, in order or treat a particular disease or condition affecting the patient. The present pharmaceutical combinations can be formulated in suitable pharmaceutical compositions for enteral or further parenteral administration, such as film (e.g. sugar-) coated tablets, tablets, capsules or suppositories, sachets or ampoules. If not indicated otherwise, these are prepared in a manner known per se, for example by means of various conventional mixing, comminution, direct compression, granulating, (e.g. sugar-) coating, dissolving, lyophilizing processes, or fabrication techniques readily apparent to those skilled in the art. It will be appreciated that the unit content of a combination partner (therapeutic agent) contained in an individual dose of each dosage form need not in itself constitute an effective amount since the necessary effective amount may be reached by administration of a plurality of dosage units. The pharmaceutical composition may contain, from about 0.1% to about 99.9%, preferably from about 0.1% to about 60%, of any one single or each of the therapeutic agent(s), in case of a fixed combination summing up to an amount of 0.2% to 70%. One of ordinary skill in the art may select one or more of the aforementioned carriers with respect to the particular desired properties of the dosage form by routine experimentation and without any undue burden. The amount of each carriers used may vary within ranges conventional in the art. The following references disclose techniques and excipients used to formulate oral dosage forms: The Handbook of Pharmaceutical Excipients, 4th edition, Rowe et al., Eds., American Pharmaceuticals Association (2003); and Remington: the Science and Practice of Pharmacy, 20th edition, Gennaro, Ed., Lippincott Williams & Wilkins (2003). These optional additional conventional carriers may be incorporated into the oral dosage form either by incorporating the one or more conventional carriers into the initial mixture before or during granulation or by combining one or more conventional carriers with granules comprising the combination of therapeutic agents or individual therapeutic agents of the combination of therapeutic agents in the oral dosage form. In the latter embodiment, the combined mixture may be further blended, e.g., through a V-blender, and subsequently compressed or molded into a tablet, for example a monolithic tablet, encapsulated by a capsule, or filled into a sachet.

Where “%” is mentioned, this refers to weight percent if not indicated otherwise.

Specific formulations relate to film coated tablets of the active ingredients in the separate or further fixed combined preparations used in the present combination treatment(s).

The pharmaceutical combinations and compositions of the invention may include a “therapeutically effective amount” or “effective amount” of a RAF inhibitor, especially naporafenib, and a MEK-inhibitor, especially trametinib. The term “pharmaceutically effective amount”, “therapeutically effective amount” or “clinically effective amount” of a combination of therapeutic agents is an amount sufficient, at dosages and for periods of time necessary, to provide an observable or clinically significant improvement over the baseline of clinically observable signs and symptoms of the disorders treated with the combination. A therapeutically effective amount may vary according to factors such as the disease state, age, sex, and weight of the individual. A therapeutically effective amount is also one in which any toxic or detrimental effects of the therapeutic agents are outweighed by therapeutically beneficial effects. A “therapeutically effective dosage” preferably modulates a measurable parameter, such as tumor growth rate or disease progression in a desired manner. The ability of a therapeutic agent to modulate a measurable parameter can be evaluated in an animal model system predictive of efficacy in human sarcomas to help establish suitable dosing levels and schedules. Alternatively, this property of a composition can be evaluated by examining the ability of the compound to modulate an undesired parameter by using in vitro assays known to the skilled practitioner or especially in clinical studies with patients, especially in need of treatment.

Preferably, the following dosages are used in combinations for a RAF inhibitor, especially naporafenib, or a pharmaceutically acceptable salt, hydrate and/or solvate thereof, and a MEK inhibitor, especially trametinib, or a pharmaceutically acceptable salt, hydrate and/or solvate thereof, according to the present invention in the case of treatment of human patients, in each case referring to the free form of the respective API:

A unit dosage of the active ingredients may be administered once daily, or twice daily, or three times daily, or four times daily, in sum yielding the appropriate daily dosage, e.g. as indicated below, with the actual dosage and timing of administration determined by criteria such as the patient's age, weight, and gender; the extent and severity of the cancer to be treated; and the judgment of a treating physician. Oral dosage forms, especially (preferably film-coated) tablets, are preferred.

Where doses or dosages are mentioned herein, the amount referred to refers to the amount of the therapeutic agent (active ingredient, here in free form). For example, when a 2 mg dosage of trametinib is administered, and trametinib is administered in a tablet containing trametinib dimethyl sulfoxide, the tablet will contain trametinib dimethyl sulfoxide equivalent to 2 mg free form of trametinib. The corresponding reasoning is valid for naporafenib monohydrate.

The RAF inhibitor is preferably administered orally. In a preferred embodiment, the RAF inhibitor, especially naporafenib, or a pharmaceutically acceptable salt thereof, respectively, is administered at a total daily dose (TTD) of from about 100 mg to about 1200 mg. The total daily dose of naporafenib is administered in one or more dosage units daily. Preferably the total daily dose of naporafenib is administered twice daily. Thus, naporafenib may be administered from about 100 mg to about 600 mg twice daily.

In some embodiments, naporafenib is administered (preferably orally) once daily (QD) at a dose of about, respectively, 100 mg, 125 mg, 150 mg, 175 mg, 200 mg (preferred), 225 mg, 250 mg, 275 mg, 300 mg, 325 mg, 350 mg, 375 mg or especially 400 mg.

In a particular embodiment, naporafenib, or a pharmaceutically acceptable salt, hydrate and/or solvent thereof, is administered orally at a dosage of about 200 mg or 400 mg twice daily (BID or bid), based on free form.

The MEK inhibitor as part of the combination or combination treatment according to the present invention will be administered to a subject in need thereof in a therapeutically effective amount.

In a preferred embodiment, the MEK inhibitor trametinib, or a pharmaceutically acceptable salt, hydrate or solvate thereof, administered as part of the combination according to the present invention in a subject in need thereof will be an amount selected from about 0.125 mg to about 10 mg per day; suitably, the amount will be selected from about 0.5 mg to about 2 mg per day; suitably, the amount will be about 1 mg or 2 mg per day. In a preferred embodiment, trametinib, trametinib, or a pharmaceutically acceptable salt or solvate thereof, is administered at daily dose of 0.5 mg or 1 mg per day, especially at 1 mg per day. Preferably, the dosage per day (daily dosage) is administered once daily (qd).

Most preferably, based on the free form, respectively, the Raf inhibitor, especially naporafenib, or a pharmaceutically acceptable salt, hydrate and/or solvate thereof, is administered in a dosage of 400 mg BID and the MEK inhibitor, especially trametinib, or a pharmaceutically acceptable salt, hydrate and/or solvate thereof, is administered in a dosage of 0.5 mg QD; or yet more preferably, the Raf inhibitor, especially naporafenib, or a pharmaceutically acceptable salt, hydrate and/or solvate thereof, is administered in a dosage of 200 mg BID and the MEK inhibitor, especially trametinib, or a pharmaceutically acceptable salt, hydrate and/or solvate thereof, is administered in a dosage of 1.0 mg QD.

In some embodiments, trametinib, or a pharmaceutically acceptable salt or solvate thereof, is administered orally. In one embodiment, trametinib is prepared for administration via oral delivery, and may be used in solvated form in dimethyl sulfoxide. In some embodiments, the compound is prepared in tablet form for oral administration, especially as film-coated tablet. The tablets can be produced in a variety of dosages for flexible administration.

The unit dosage of trametinib, or a pharmaceutically acceptable salt or solvate thereof, may be administered once daily, or twice daily, or three times daily, or four times daily. The total daily dose of trametinib, or a pharmaceutically acceptable salt or solvate thereof, e.g., the dimethyl sulfoxide solvate, may be administered once or further twice a day.

For example, as part of the combination therapy, Compound of formula (I), or a pharmaceutically acceptable salt thereof, e.g. the compound in monohydrate or preferably free form, may be administered at a total daily dose of about 200 mg, about 300 mg, about 400 mg or about 500 mg and trametinib, e.g. in the dimethyl sulfoxide solvate form, may be administered in a total daily dose of about 0.5 or 1.0 mg. The daily dose of the Compound of formula (1) may be administered once or twice per day. Hence, a dose of about 200 mg of Compound of formula (I) may be administered twice per day and (total daily dose about 400 mg) and a dose of about 1.0 mg or about 2.0 mg of trametinib may be administered once per day. Alternatively, a dose a dose of about 200 mg of Compound of formula (I) may be administered twice per day and (total daily dose about 400 mg) and a dose of about 0.5 or about 1.0 mg of trametinib may be administered twice per day.

A further benefit may be that lower doses of the therapeutic agents of the combination of the invention can be used, for example, such that the dosages may not only often be smaller, but also may be applied less frequently, or can be used in order to diminish the incidence of side-effects observed with one of the combination partners alone. This is in accordance with the desires and requirements of the patients to be treated.

The term “jointly therapeutically active” or “joint therapeutic effect” as used herein means that the therapeutic agents can be given jointly, separately or sequentially in such time intervals such that the patient, especially human, to be treated, still shows an (preferably synergistic) interaction (joint therapeutic effect). Whether this is the case can, inter alia, be determined by following the blood levels of the compounds, showing that both compounds are present in the blood of the human to be treated at least during certain time intervals, or by the fact that subsequent treatment leads to a better result than single treatment with only one of the active ingredients.

The term “active ingredient” or “therapeutic agent” comprises the RAF inhibitor and the MEK inhibitor as defined for combinations according to the invention embodiments.

In one embodiment, the disease or disorder amenable to or to be treated with a combination treatment according to the invention is cancer. The term “cancer” is used herein to mean a tumor selected from a broad spectrum of tumors, including all solid tumors. The cancer may be at an early, intermediate or late stage. The cancer may be locally advanced or metastatic. “Cancer” or “Tumor” are used synonymously herein and these identifiers are equivalent to “tumor disease” or “cancer disease” and, in the case of solid tumors, especially provides an advanced tumor/tumor disease.

The cancer to be treated by the combination therapy described herein may have progressed following standard of care or for whom no effective standard therapy exists.

In one embodiment the solid tumor is non-benign. In one embodiment, the solid tumor is selected from the group comprising melanoma, or more particularly any solid tumor other than melanoma, such a tumor selected from the group consisting of lung cancer, especially non-small-cell lung cancer (NSCLC), colorectal cancer (CRC), such as colorectal adenocarcinoma, pancreatic cancer, such as pancreatic ductal adenocarcinoma (PADC), or further from head and neck cancer, such as head and neck squamous cell carcinoma, bladder urothelial carcinoma, MUTYH-associated polyposis (MAP), cervical cancer and ovarian cancer, an esophageal cancer, a kidney cancer, a mesothelioma, a thyroid cancer, a prostate cancer, a glioblastoma, a cervical cancer, a thymic carcinoma, a Merkel cell cancer, or further a sarcoma, e.g. rhabdomyosarcoma.

In one embodiment, the tumor is melanoma.

In one embodiment, the tumor is a lung cancer, e.g. non-small cell lung cancer (NSCLC).

In one embodiment, the tumor is a colorectal cancer.

In one embodiment, the tumor is a pancreatic cancer.

In one embodiment, the tumor is a head and neck cancer.

In one embodiment, the tumor is a bladder urothelial carcinoma.

In one embodiment, the proliferative disease is a colorectal cancer (CRC) including MUTYH-associated polyposis (MAP).

In one embodiment, the tumor is a cervical cancer.

In one embodiment, the tumor is an ovarian cancer.

In one embodiment (in particular for G13R mutation), the tumor is a rhabdomyosarcoma.

The solid tumor to be treated includes NRAS-mutant melanoma. Where melanoma is mentioned, this relates especially to “NRAS-mutant melanoma”. Where “NRAS-mutant melanoma” is mentioned, this relates especially to ocular or cutaneous melanoma-unless context clearly dictates otherwise. Preferably this term refers to cutaneous melanoma, in particular to unresectable and/or metastatic cutaneous melanoma.

The present invention provides a pharmaceutical combination for use in treating melanoma in a patient wherein the melanoma has been previously treated, e.g. by surgical removal, or other therapy and progressed after such therapy.

The melanoma to be treated by the combination may be melanoma which is refractory or resistant to previous treatment with another therapy. The combination of the invention may therefore be useful as second-line, third-line or fourth-line treatment of melanoma.

Prior treatment for melanoma patients includes:
treatment with talimogene laherparepvec (also known by the tradename T-Vec, Imlygic or Oncovex) which is a biopharmaceutical drug to treat unresectable melanoma and which is injected directly into a subset of metastatic lesions;
standard care chemotherapy (e.g., dacarzabine);
treatment with a cytotoxic agent such as a nitrosurea and/or mitomycin C;
immunotherapy (e.g., pembrolizumab, ipilimumab, or nivolumab and combinations thereof);
targeted therapy (e.g., dabrafenib and trametinib, vemurafenib and cobimetinib, and encorafenib and binimetinib).

The patient to be treated thus includes a patient suffering from NRAS-mutant melanoma, especially a patient who has received previous therapy including standard care chemotherapy (e.g., dacarzabine), immunotherapy (e.g., pembrolizumab, ipilimumab, or nivolumab and combinations thereof), targeted therapy (e.g., dabrafenib and trametinib, vemurafenib and cobimetinib, and encorafenib and binimetinib) and who has progressed on previous therapy. The patient may be a patient suffering from NRAS-mutant melanoma especially where the melanoma is cutaneous melanoma which is unresectable and/or metastatic.

In another embodiment, the NRAS-mutant melanoma is resistant or refractory to standard of care.

In another embodiment, the NRAS-mutant melanoma is resistant or refractory to standard of care with dacarbazine.

In another embodiment, the melanoma is resistant or refractory to treatment with a cytotoxic agent such as a nitrosurea and/or mitomycin C.

In another embodiment, the melanoma is resistant or refractory to treatment with immunotherapy treatment including therapy with one or more immune checkpoint inhibitors.

Thus in one embodiment, the melanoma to be treated by a combination of the present invention is NRAS-mutant melanoma which is resistant to immunotherapeutic PD-1 (Programmed Cell death 1 receptor) or PD-L1 (the ligand of PD-1) treatment, alone or in combination with an anti-CTLA4 (cytotoxic T-lymphocyte-associated protein) antibody (e.g. ipilimumab). See e.g. Tsai et al, Human Vaccines & Immunotherapeutics 10:11, 3111--3116; November 2014.

Thus in one embodiment, the melanoma (including NRAS-mutant melanoma) to be treated is resistant or refractory to treatment with one or more therapeutic agents selected from ipilimumab, spartalizumab, nivolumab, pembrolizumab, pidizilumab, BMS-9365559, MEDI4736, and MSB0010718C.

Thus, the melanoma to be treated by the combination of the present invention includes NRAS-mutant melanoma which is resistant to anti-PD-1 monotherapy (such as pembrolizumab or nivolumab) or a combination of anti-PD-1 agent with ipilimumab.

Genetic assessment of BRAF, NRAS and NF1-mutations in patients can be conducted according to methods known in the art, e.g., using SNaPshot or DFCI Oncopanel as described previously (Sholl L M, et al. JCI Insight 2016; 1: e87062; Zheng Z, et al., Nat Med 2014; 20:1479-84). The current iterations of both assays utilize next-generation sequencing, whereas earlier versions of SNAPshot relied on multiplex PCR. The current version of SNAPshot interrogates exons 11 and 15 of BRAF, exons 2-5 of KRAS and NRAS, and exons 1-58 of NF1. Oncopanel detects alterations involving all exons of BRAF, KRAS, NRAS, and NF1.

A subject suffering from cancer/a tumor is defined as having progressed on, or no longer responding to therapy with one or more agents, or being intolerant to with one or more agents when the cancer he or she is suffering from, has progressed. The progression of cancer may be monitored by methods well known to those in the art. For example, the progression may be monitored by way of visual inspection of the cancer, such as, by means of X-ray, CT scan or MRI or by tumor biomarker detection. For example, an increased growth of the cancer indicates progression of the cancer. Progression of cancer such as NSCLC or tumors may be indicated by detection of new tumors or detection of metastasis or cessation of tumor shrinkage. Tumor evaluations can be made based on RECIST criteria (Therasse et al 2000), New Guidelines to Evaluate the Response to Treatment in Solid Tumors, Journal of National Cancer Institute, Vol. 92; 205-16 and revised RECIST guidelines (version 1.1) (Eisenhauer et al 2009) European Journal of Cancer; 45:228-247. For example, to show a therapeutic effect, the Overall Response Rate (ORR) or any other parameter according to the Response Evaluation Criteria In Solid Tumors (RECIST v1.1) may be used.

Complete Response is defined as disappearance of all non-nodal target lesions. In addition, any pathological lymph nodes assigned as target lesions must have a reduction in short axis to <10 mm 1.

Partial Response is defined as at least a 30% decrease in the sum of diameter of all target lesions, taking as reference the baseline sum of diameters.

Other response criteria can be deduced from the following table (according to RECIST v1.1):

| Response Criteria | Evaluation of target lesions |
|---|---|
| Progressive Disease (PD): | At least a 20% increase in the sum of diameter of all measured target |
| | lesions, taking as reference the smallest sum of diameter of all target |
| | lesions recorded at or after baseline. In addition to the relative increase |
| | of 20%, the sum must also demonstrate an absolute increase of at least |
| | 5 mm ². |
| Stable Disease (SD): | Neither sufficient shrinkage to qualify for PR or CR nor an increase in |
| | lesions which would qualify for PD. |
| Unknown (UNK) | Progression has not been documented and one or more target lesions |
| | have not been assessed or have been assessed using a different method |
| | than baseline. |

The progression of tumor, tumor burden increase or decrease, and response to treatment with an inhibitor combination according to the invention may be monitored by methods well known to those in the art. Thus the progression and the response to treatment may be monitored by way of visual inspection of the tumor, such as, by means of X-ray, CT scan or MRI or by tumor biomarker detection.

Tumor progression may be determined by comparison of tumor status between time points after treatment has commenced or by comparison of tumor status between a time point after treatment has commenced to a time point prior to initiation of the relevant treatment.

The combination and combination treatment according to the invention is particularly useful for the treatment of a cancer that harbors one or more Mitogen-activated protein kinase (MAPK) pathway alterations, including a HRAS or especially KRAS-mutant tumor and/or a NRAS-mutant tumor, and in particular, a tumor expressing at least one gain-of-function mutation of RAS, especially KRAS or NRAS, as described herein, selected from codon Q61, such as Q61H, Q61K mutation, or (especially) G13R mutation, not limited to but also in case of at least one gain-of-function mutation of RAF.

In another embodiment cancer is resistant or refractory to treatment with a MEK inhibitor, e.g. trametinib.

The present invention, as was already mentioned, rests on the important finding that certain RAS mutations, especially at codon Q61 or mutation G13R, are appropriate biomarkers for the amenability of tumor patients for combined treatment with a RAF and a MEK inhibitor.

Where a RAS, e.g. a KRAS, HRAS or NRAS (codon) mutation is mentioned herein, this relates, on the one hand, to a corresponding point mutation in the polynucleotide (e.g. gene, cDNA or mRNA), on the other hand to the correspondingly mutated protein with the altered amino acid sequence at the mentioned position (Q61 or G13R).

Appropriate assays, methods and agents (especially binding reagents that bind to the biomarker to be identified) allowing screening for, examining for or identifying the presence of RAS, e.g. KRAS, codon Q61or G13R mutations are in principle known and available to the person skilled in the art.

A “KRAS activating mutation” is any mutation of the KRAS gene (i.e., a nucleic acid mutation) or Kras protein (i.e., an amino acid mutation) that results in aberrant Kras protein function associated with increased and/or constitutive activity by favoring the active GTP-bound state of the Kras protein. The mutations are at conserved sites that favor GTP binding and constitutively active Kras protein. A mutation of use according to the present invention is at certain codons of the KRAS gene coding for KRAS comprising KRAS-G13R (especially preferred), or KRAS-Q61E, KRAS-Q61P, KRAS-Q61H, KRAS-Q61K (preferred), KRAS-Q61L (preferred) or KRAS-Q61R (preferred).

An “NRAS activating mutation” is any mutation of the NRAS gene (i.e., a nucleic acid mutation) or Nras protein (i.e., an amino acid mutation) that results in aberrant Nras protein function associated with increased and/or constitutive activity by favoring the active GTP-bound state of the Nras protein. The mutation is at conserved sites that favor GTP binding and constitutively active Nras protein. A mutation of use according to the invention is at certain codons of the NRAS gene coding for KRAS comprising NRAS-G13R mutation (especially preferred), or NRAS-Q61E, NRAS-Q61P, NRAS-Q61H, NRAS-Q61K (preferred), NRAS-Q61L (preferred) or NRAS-Q61R (preferred) mutations.

Methods of identifying RAS codon Q61or RAS G13R mutations are known in the art, e.g. by using “next-generation sequencing.” An agent or probe capable of identifying RAS codon Q61or RAS G13R mutation are known in the art and may be preferably selected from an antibody and, a polynucleotide, each capable of binding to the site of the mutation, either in the expressed protein or preferably in the gene DNA, cDNA or mRNA coding for it.

The term “antibody” herein is used in the broadest sense and encompasses various antibody structures, including but not limited to monoclonal antibodies, polyclonal antibodies, multispecific antibodies (e.g., bispecific antibodies), single chain antibodies and antibody fragments so long as they exhibit the desired antigen-binding activity.

“Polynucleotide” or “nucleic acid” (the terms being used as synonyms herein) refers to a polymer of nucleotides of any length and include one or both of DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, the term includes, without limitation, single- and double-stranded DNA, DNA including single- and double-stranded regions, single- and double-stranded RNA, and RNA including single- and double-stranded regions, hybrid molecules comprising DNA and RNA that may be single stranded or, more typically, double-stranded or include single- and double-stranded regions. For a nucleic acid or polynucleotide mutation of use according to the invention (=one that allows to make a prognosis on the amenability of a tumor (and thus patient) for successful combined treatment with a RAF inhibitor and a MEK inhibitor, DNA and RNA are preferred, especially cDNA or mRNA.

If present, especially in the case of a polynucleotide (especially oligonucleotide) or nucleic acid used according to the invention for identifying a mutation of use according to the invention (e.g. as a probe or primer) in a RAS DNA or RNA from a tumor sample, modification to the nucleotide structure may be imparted before or after assembly of the polymer. The sequence of nucleotides may be interrupted by non-nucleotide components. Such a polynucleotide may be further modified after synthesis, such as by conjugation with an (e.g fluorescence or isotope) label. Other types of modifications include, for example, “caps,” substitution of one or more of the naturally-occurring nucleotides with an analog, internucleotide modifications, for example, those with uncharged linkages (e.g., methyl phosphonates, phosphotriesters, phosphoamidates or carbamates) and/or with charged linkages (e.g., phosphorothioates or phosphorodithioates), those containing (covalently or otherwise bound) attached moieties, for example, proteins (e.g., nucleases, toxins, antibodies, signal peptides or poly-L-lysine), those with intercalators (e.g., acridine or psoralen), those containing chelators (e.g., metals, radioactive metals, boron or oxidative metals), those containing alkylators, those with modified linkages (e.g., alpha anomeric nucleic acids), as well as unmodified forms of the polynucleotide(s). Any one or more of the hydroxyl groups ordinarily present in the sugars of the nucleotide backbone may be replaced, for example, by phosphonate groups, phosphate groups, protected by customary protecting groups, or activated to prepare additional linkages to additional nucleotides, or may be conjugated to solid or semi-solid supports. The terminal OH can be phosphorylated or substituted with amines or organic capping group moieties of from 1 to 20 carbon atoms. Other hydroxyls may also be derivatized to standard protecting groups. These polynucleotides can also contain analogous forms of ribose or deoxyribose sugars that are generally known in the art, including, for example, 2′-Omethyl-, 2′-O-allyl-, 2′-fluoro-, or 2′-azido-ribose, carbocyclic sugar analogs, a-anomeric sugars, epimeric sugars such as arabinose, xylose or lyxose, pyranose sugars, furanose sugars, sedoheptuloses, acyclic analogs, and a basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or aralkyl. Not all linkages in a polynucleotide need be identical. Such a polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are typically, but not necessarily, less than about 250 nucleotides in length, e.g. from 2 to 80, such as from 2 to 50 nucleotides in length. Oligonucleotides (especially where used as probes, e.g. primers) may be synthetic.

The term “primer” refers to a single-stranded oligo- or polynucleotide that is capable of hybridizing to a nucleic acid (especially a DNA or RNA from a tumor sample) and allowing polymerization of a complementary nucleic acid, normally by providing a free 3′—OH group.

The term “biomarker” as used herein refers to any part of a molecule in a patient that provides testimony of RAS G13R (especially preferred), Q61E, Q61P, Q61H, Q61K, Q61Lor Q61R mutations. The biomarker may be a predictive biomarker and serve as an indicator of the likelihood of amenability (=sensitivity or benefit) of a patient having a solid tumor disease with a tumor harboring one of the mentioned RAS mutations, which allows to predict that the patient is amenable to treatment with a RAF, e.g. a Raf inhibitor, for example, a Raf dimer, in combination with a MEK inhibitor. Biomarkers include, but are not limited to, polynucleotides (e.g., DNA and/or RNA (e.g., mRNA)), polynucleotide copy number alterations (e.g., DNA copy numbers), polypeptides (e.g. the corresponding Ras polypeptides), modified polypeptides or polynucleotides (e.g., with post-translational modifications). In some embodiments, as referenced above or below, a biomarker is a gene (e.g., the KRAS or NRAS gene) carrying the mutation of interest.

Especially in the case of DNA or RNA to be examined for the presence of the biomarker (the mutation of interest according to the present invention) from a solid tumor or a patient, after isolation of the DNA or RNA, preferably an amplification of at least the region of its sequence (the temülate sequence) that contains a mutation of interest is conducted.

Prospective or retrospective genetic assessment of RAS mutations in patients can be conducted according to methods known in the art, e.g., using SNapshot or DFCI Oncopanel as described previously (Sholl L M, et al. JCI Insight 2016; 1: e87062; Zheng Z, et al., Nat Med 2014; 20:1479-84). The current iterations of both assays utilize next-generation sequencing (see e.g. Boland G M, et al. Clinical next generation sequencing to identify actionable aberrations in a phase I program. Oncotarget. 2015; 6 (24): 20099-20110), whereas earlier versions of SNAPshot relied on multiplex PCR. The current version of SNAPshot interrogates exons 2-5 of KRAS and NRAS, Oncopanel detects alterations involving all exons of RAS, especially KRAS, NRAS.

Amplifying” or “amplification” as used herein generally refers to the process of producing multiple copies of a desired nucleotide sequence. A “copy” normally has (but further not necessarily needs to have) perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

A preferred variant of identifying a mutation of interest according to the invention includes a polymerase chain reaction step.

The (herein most important and preferred) technique of “polymerase chain reaction” or “PCR” as used herein generally refers to a procedure wherein minute amounts of a specific nucleic acid segment, RNA and/or DNA, are amplified as described, for example, in U.S. Pat. No. 4,683,195, Schuller M., et al., (eds.), “PCR for Clinical Microbiology—an Australian and International Perspective, Springer 2010, B. Y. Chan and H. W. Janes (eds.), PCR Cloning Protocols, 2^{nd} ed., Humana press 2002, or Mullis et al., Cold Spring Harbor Symp. Quant. Biol. 51:263 (1987) and Erlich, ed., PCR Technology, (Stockton Press, NY, 1989). Generally, sequence information from the region of interest is required, such that oligonucleotide primers can be designed, which will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. Any nucleic acid polymerase reaction method for amplifying a nucleic acid test sample, comprising the use of a known nucleic acid (DNA or RNA) as a primer and utilizing a nucleic acid polymerase to amplify or generate a specific piece of nucleic acid or to amplify or generate a specific piece of nucleic acid which is complementary to a particular nucleic acid may be used.

The term “multiplex-PCR” refers to a single PCR reaction carried out on nucleic acid obtained from a single source (e.g., a solid tumor sample from one individual) using more than one primer set for the purpose of amplifying two or more DNA sequences in a single reaction.

“Quantitative real-time polymerase chain reaction” or “qRT-PCR” refers to a special form of PCR allowing measuring the amount of PCR product at each step in a PCR reaction if desired. This technique has been described in various publications including, for example, Cronin et al., Am. J. Pathol. 164 (1): 35-42 (2004) and Ma et al., Cancer Ce//5:607-616 (2004).

The disclosed methods and assays provide for convenient, efficient, and potentially cost-effective means to obtain data and information useful in assessing appropriate or effective therapies for treating 40 patients. For example, a patient can provide a tumor sample as tissue sample (e.g., a tumor biopsy) before combined treatment with a RAF inhibitor, e.g. a RAF inhibitor, in combination with a MEK inhibitor is considered as a treatment option, and the sample can be examined by way of various in vitro assays to determine whether the patient's cells are sensitive to said combined treatment.

In any of the preceding methods, identification of the particular mutational status of the HRAS, or especially KRAS and/or NRAS gene, mRNA, or protein product in a sample obtained from the individual can be performed by any one or more methods well known to a person skilled in the art. For example, identification of the mutation can be accomplished by cloning of the HRAS, especially KRAS and/or especially NRAS gene, or portion thereof, and sequencing it using techniques well known in the art. Alternatively, the gene sequences can be amplified from genomic DNA or cDNA, e.g. using PCR, and the product sequenced. Several non-limiting methods for analyzing a patient's DNA for mutations at a given genetic locus are described below.

DNA microarray technology, e.g., DNA chip devices and high-density microarrays for high throughput screening applications and lower-density microarrays, may be used, e.g. for DNA microarray hybridization. Additional methods include interference RNA microarrays and combinations of microarrays and other methods such as laser capture microdissection (LCM), comparative genomic hybridization (CGH) and chromatin immunoprecipitation (ChiP). See, e.g., He et al. (2007) Adv. Exp. Med. Biol. 593:117-133 and Heller (2002) Annu. Rev. Biomed. Eng. 4:129-153.

Other methods include PCR, xMAP, invader assay, mass spectrometry, and pyrosequencing (Wang et al. (2007) 593:105-106).

Another detection method is allele specific hybridization using probes overlapping the polymorphic site and having about 5, or alternatively 10, or alternatively 20, or alternatively 25, or alternatively 30 nucleotides around the region where the mutation site is looked for. For example, several probes capable of hybridizing specifically to a particular mutational variant (e.g., KRAS-G13R, which corresponds to a KRAS c.38G>A nucleotide substitution point mutation) are attached to a solid phase support, e.g., a “chip.”

Oligonucleotides can be bound to a solid support by a variety of processes, including lithography. Mutation detection analysis using these chips comprising oligonucleotides, also termed “DNA probe arrays” is described e.g., in Cronin et al. (1996) Human Mutation 7:244.

In other detection methods, it is necessary to first amplify at least a portion of the gene prior to identifying the mutational variant. Amplification can be performed, e.g., by PCR and/or LCR or other methods well known in the art.

In some cases, the presence of the specific mutation in DNA from a subject can be shown by restriction enzyme analysis. For example, the specific mutation can result in a nucleotide sequence comprising a restriction site which is absent from the nucleotide sequence of another mutational variant or the wild-type version of the gene.

In a further embodiment, protection from cleavage agents (such as a nuclease, hydroxylamine, or osmium tetroxide and with piperidine) can be used to detect mismatched bases in RNA/RNA, DNA/DNA or RNA/DNA heteroduplexes (see, e.g., Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene with a sample nucleic acid, e.g., RNA or DNA, obtained from a tumor sample. The double-stranded duplexes are treated with an agent which cleaves single-stranded regions of the duplex such as duplexes formed based on basepair mismatches between the control and sample strands. For instance, RNNDNA duplexes can be treated with RNase and DNNDNA hybrids treated with SI nuclease to enzymatically digest the mismatched regions. Alternatively, either DNA/DNA or RNNDNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. (1988) Proc. Natl. Acad. Sci. USA 85:4397; Saleeba et al. (1992) Meth. Enzymol. 217:286-295.

Furthermore, alterations in electrophoretic mobility may also be used to identify the particular allelic variant. For example, single-strand conformation polymorphism (SSCP) may be used to detect differences in electrophoretic mobility between mutant and wild type nucleic acids (Orita et al. (1989) Proc Natl. Acad. Sci USA 86:2766; Cotton (1993) Mutat. Res. 285:125-144 and Hayashi (1992) Genet. Anal. Tech. Appl. 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another embodiment, the subject method utilizes heteroduplex analysis to separate double-stranded heteroduplex molecules on the basis of changes in electrophoretic mobility (Keen et al. (1991) Trends Genet. 7:5).

The identity of the mutational variant may also be determined by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys. Chem. 265:1275).

Examples of techniques for detecting differences of at least one nucleotide between two nucleic acid molecules (e.g., DNA or RNA molecules) include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is 40 found (Saiki et al. (1986) Nature 324:163); Saiki et al. (1989) Proc. Natl. Acad. Sci. USA 86:6230). Such allele specific oligonucleotide hybridization techniques may be used for the detection of the nucleotide changes in the polymorphic region of the gene. For example, oligonucleotides having the nucleotide sequence of the specific allelic variant are attached to a hybridizing membrane and this membrane is then hybridized with labeled sample nucleic acid. Analysis of the hybridization signal will then reveal the 5 identity of the nucleotides of the sample nucleic acid.

Alternatively, allele specific amplification technology, which depends on selective PCR amplification, may be useful in conjunction with the instant invention.

Oligonucleotides used as primers for specific amplification may carry the allelic variant of interest in the center of the molecule (so that amplification depends on differential hybridization) (Gibbs et al. (1989) Nucl. Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition, it may be desirable to introduce a novel restriction site in the region of the mutation to create cleavage-based detection (Gasparini et al. (1992) Mot. Cell. Probes 6:1).

In another embodiment, identification of the mutational variant is carried out using an oligonucleotide ligation assay (OLA), as described, e.g., in U.S. Pat. No. 4,998,617 and in Laridegren, U. et al. Science 241:1077-1080 (1988). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson et al. have described a nucleic acid detection assay that combines attributes of PCR and OLA (Nickerson, D. A. et al. (1990) Proc. Natl. Acad. Sci. USA 87:8923-8927). In this method, PCR is used to achieve the exponential amplification of target DNA, which is then detected using OLA.

Described are methods for detecting a single-nucleotide mutation in a given gene, for example, a RAS gene, such as the KRAS gene or the NRAS gene or the HRAS gene. Because single-nucleotide changes are flanked by regions of invariant sequence, their analysis requires no more than the determination of the identity of the single variant nucleotide, and it is unnecessary to determine a complete gene sequence for each patient. Several methods have been developed to facilitate the analysis of such mutations.

Single-base mutations can be detected by using a specialized exonuclease-resistant nucleotide, as disclosed, e.g., in U.S. Pat. No. 4,656,127. According to the method, a primer complementary to the mutated sequence immediately 3′ to the mutated site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the mutated site of the target molecule was complementary to that of the nucleotide derivative used in the reaction. This method has the advantage that it does not require the determination of large amounts of extraneous sequence data.

A solution-based method may also be used for determining the identity of the nucleotide of the mutated site (WO 91/02087). As above, a primer is employed that is complementary to mutated sequence(s) immediately 3′ to a mutated site. The method determines the identity of the nucleotide of that site using labeled dideoxynucleotide derivatives, which, if complementary to the nucleotide of the mutated site, will become incorporated onto the terminus of the primer.

An alternative method is described in WO 92/15712. This method uses mixtures of labeled terminators and a primer that is complementary to the sequence 3′ to a mutated or polymorphic site. The labeled terminator that is incorporated is thus determined by, and complementary to, the nucleotide present in the mutated site of the target molecule being evaluated. The method is usually a heterogeneous phase assay, in which the primer or the target molecule is immobilized to a solid phase.

Many other primer-guided nucleotide incorporation procedures for assaying mutated sites in DNA have been described (Komher, J. S. et al. (1989) Nucl. Acids. Res. 17:7779-7784; Sokolov, B. P. (1990) Nucl. Acids Res. 18:3671; Syvanen, A.-C., et al. (1990) Genomics 8:684-692; Kuppuswamy, M. N. et al. (1991) Proc. Natl. Acad. Sci. USA 88:1143-1147; Prezant, T. R. et al. (1992) Hum. Mutat. 1:159-164; Ugozzoli, L. et al. (1992) GA TA 9:107-112; Nyren, P. et al. (1993) Anal. Biochem. 208:171-175). These methods all rely on the incorporation of labeled deoxynucleotides to discriminate between bases at a mutated site.

In general, the presence and/or amount of a biomarker gene described herein can be analyzed by a number of methodologies, including those described above, as well as many others known in the art and understood by the skilled artisan, such as Southern analysis, Northern analysis, whole genome sequencing, polymerase chain reaction (PCR) (including quantitative real time PCR (qRT-PCR), Next Generation Sequencing and other amplification type detection methods, such as, for example, branched DNA, SISBA, TMA and the like), RNA-Seq, microarray analysis, Nanostring, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology,

### Units 2 (Northern Blotting), 4 (Southern Blotting), 15 (Immunoblotting), and 18 (PCR Analysis).

Multiplexed immunoassays such as those available from Rules Based Medicine or Meso Scale Discovery (“MSD”) may also be used.

In addition, the presence and/or amount of a biomarker protein (i.e., gene product) described herein can be analyzed by a number of methodologies, including immunohistochemistry (“IHC”), Western blot analysis, immunoprecipitation, spectroscopy, molecular binding assays, HPLC, enzyme-linked immunosorbent assay (ELISA), enzyme-linked immunofiltration assay (ELIFA), fluorescence activated cell sorting (“FAGS”), MassARRAY, proteomics, quantitative blood based assays (e.g., serum ELISA), biochemical enzymatic activity assays, in situ hybridization, fluorescence in situ hybridization (FISH), and protein sequencing. In certain instances, the method comprises contacting a biological sample from the individual with antibodies that specifically bind to a protein biomarker described herein under conditions permissive for binding of the biomarker, and detecting whether a complex is formed between the antibodies and biomarker. Such a method may be an in vitro or in vivo method. In some instances, the protein expression level of the biomarker (e.g., KRAS-G13R protein or an NRAS protein having an activating mutation, such as one described herein above) is determined in tumor cells (e.g., from a biopsy).

Moreover, it will be understood that any of the above methods for detecting mutations in a gene or gene product can also be used to monitor the course of treatment or therapy (e.g., combined treatment including a RAF inhibitor, e.g. a Raf inhibitor and a MEK inhibitor).

The methods described herein may be performed, for example, by utilizing pre-packaged diagnostic kits, such as those described below, comprising at least one probe or primer nucleic acid, which may be conveniently used, e.g., to determine whether a subject is likely to benefit from a combined treatment including a RAF inhibitor, e.g., a Raf inhibitor and a MEK or P13K inhibitor.

In all methods of screening described above or referenced herein, a mutation in one or more RAS genes (e.g., HRAS, KRAS or NRAS), or a protein product thereof, can generally be identified by determining a nucleic acid sequence (e.g., DNA or RNA sequence) or protein sequence (i.e., amino acid sequence) in a tumor sample obtained from an individual (=a patient) and comparing the sequence to a reference sequence (e.g., a wildtype sequence). In certain instances, a reference level, reference sample, reference cell, reference tissue, control sample, control cell, or control tissue is a single sample or a combination of multiple samples from the same subject or individual that are obtained at one or more different time points than when the test sample is obtained. For example, a reference level, reference sample, reference cell, reference tissue, control sample, control cell, or control tissue is obtained at an earlier time point from the same subject or individual than when the test sample is obtained. Such reference level, reference sample, reference cell, reference tissue, control sample, control cell, or control tissue may be useful if the reference sample is obtained during initial diagnosis of cancer and the test sample is later obtained when the cancer becomes metastatic.

Other features, objects, and advantages of the invention will be apparent from the description and drawings, and from the claims.

For further details regarding the data and information represented in the Figures also the Examples are referred to.

The following Examples illustrate the invention described above; they are not, however, intended to limit the scope of the invention in any way. The beneficial effects of the pharmaceutical combination of the present invention can also be determined by other test models known as such to the person skilled in the pertinent art.

### Examples

The Examples below are set forth to aid in the understanding of the invention but are not intended, and should not be construed, to limit its scope in any way.

### Example 1: Analysis of Data Obtained from Study CLXH254X2102 (Cf. https://clinicaltrials.gov/ct2/show/NCT02974725)

As of May-2021, 94 patients with KRAS/BRAF mutant non-small cell lung cancer and NRAS mutant melanoma have been treated, in study CLXH254X2102, with naporafenib on a bid schedule in combination with trametinib on a qd schedule at the following dose levels: 200 mg naporafenib and 1 mg trametinib (39 patients), 400 mg naporafenib and 1 mg trametinib (5 patients), 200 mg naporafenib and 0.5 mg trametinib (7 patients), 400 mg naporafenib and 0.5 mg trametinib (37 patients) and 400 mg naporafenib and 1 mg trametinib 2 weeks on, 2 weeks off (6 patients). The RDEs for the combination of naporafenib with trametinib were determined as naporafenib 400 mg bid+trametinib 0.5 mg qd and naporafenib 200 mg bid+trametinib 1 mg qd.

As of 1 Sep. 2021, efficacy data for NRAS-mutant melanoma were available in 30 patients in the expansion part; 15 were treated with naporafenib 200 mg bid and trametinib Img qd and 15 patients were treated with naporafenib 400 mg bid and trametinib 0.5 mg qd. Overall, patients received up to 7 prior lines of therapy, 6 (40%) patients received one prior line in the 200 mg/1 mg group while 4 (26.7%) patients received up to one prior line in the 400 mg/0.5 mg group. The confirmed ORR was 46.7% vs 13.3% and mPFS 5.52 vs 4.44 months in in the 200 mg/1 mg and 400 mg/0.5 mg groups respectively. Only 3 of the 30 NRAS-mutant melanoma patients did not have a confirmed Q61 or G13R codon mutation; there were no confirmed complete or partial responses for these 3 patients. See Table 1:

TABLE 1
Best overall response per RECIST 1.1 based on Investigator's assessment with confirmation by cohort,
LXH254 (LXH) + trametinib (TMT) NRAS Melanoma expansion phase patients Full analysis set

| | LXH200 mg | LXH400 mg | |
|---|---|---|---|
| | BID + TMT | BID + TMT | All |
| | 1 mg QD | 0.5 mg QD | Patients |
| | N = 15 | N = 15 | N = 30 |
| | n (%) | n (%) | n (%) |
| Best overall response | | | 0 |
| Complete Response (CR) | 0 | 0 | |
| Partial Response (PR) | 7 (46.7) | 2 (13.3) | 9 (30.0) |
| Stable Disease (SD) | 5 (33.3) | 8 (53.3) | 13 (43.3) |
| Progressive Disease (PD) | 3 (20.0) | 3 (20.0) | 6 (20.0) |
| Non-CR/Non-PD (NCRNPD) | 0 | 0 | 0 |
| Unknown (UNK) | 0 | 2 (13.3) | 2 (6.7) |
| Overall Response Rate (ORR: CR + PR) | 7 (46.7) | 2 (13.3) | 9 (30.0) |
| 95% CI ORR | (21.3, 73.4) | (1.7, 40.5) | (14.7, 49.4) |
| Disease Control Rate (DCR: CR + PR + SD) | 12 (80.0) | 10 (66.7) | 22 (73.3) |
| 95% CI ORR | (51.9, 95.7) | (38.4, 88.2) | (54.1, 87.7) |

N: The total number of subjects in the treatment group (denominator for percentage (%) calculation)
n: Number of patients who are at the corresponding category.
Bid = twice a day
Qd = once daily
ORR = Objective Response Rate
DCR = Disease Control Rate
DOR = Duration of Response
mDOR = man Duration of Response
mPFS = mean Progression Free Survival
CR = Complete Remission
PR = Partial Remission
SD = Stable Disease
mOS = mean overall survival
OSR = overall survival rate
The 95% CI for the frequency distribution of each variable were computed using Clopper-Pearson.

As of 24 Nov. 2021, efficacy data is also available for 49 patients with KRAS-mutant NSCLC; 27 were treated with naporafenib 200 mg bid and trametinib 1 mg qd and 22 patients were treated with naporafenib 400 mg bid and trametinib 0.5 mg qd. Naporafenib in combination with trametinib also showed greater efficacy in KRAS-mutant NSCLC patients with a codon Q61 or G13R mutation; 3 out of 4 NSCLC patients with tumors harboring codon Q61 or G13R KRAS mutations responded (CR or PR) to the naporafenib in combination with trametinib, versus 1 out of 45 NSCLC patients with tumors harboring KRAS mutations other than codon Q61 or G13R mutations.

This unpublished data, along with the pre-clinical evidence described above in the Background Section as well as clinical data with belvarafenib, another Raf inhibitor (see Example X below), makes it plausible that codon Q61/G13R pan-RAS mutations represent a predictive biomarker of response to naporafenib and trametinib combination. One hypothesis is that the activity of naporafenib in combination with trametinib seen in NRAS mutant melanoma may be due to the natural enrichment of codon Q61 mutations rather than to the presence of NRAS mutation itself. The presence of mutations were identified using next-generation sequencing (NGS).

### Example 2: Similarity of Effects on RAS for G13R Mutation Versus Codon Q61 Mutations

In a molecular dynamic simulation analysis performed at Novartis, G13R was found to overlap with Q61 and dynamically behaves similarly and presumably adopts similar conformation/pauses (RAF-1 proficient) as Q61.

Molecular dynamics simulations were performed with the Amber20 package (Case et al., AMBER 2020, University of California, San Francisco, 2020). The x-ray crystal structure of GMPPNP bound KRAS-G13D (PDB code 6XGV) was used for G13R simulations. The x-ray crystal structure of GMPPNP bound KRAS-Q61R (PDB code 6XGU) was used for Q61R simulations. The CCG MOE molecular editor was used to prepare the starting coordinates and in the case of G13R, mutate D13 to R13 Inc., C.C.G. “Molecular Operating Environment (MOE), 101 Sherbooke St. West, Suite #910, Montreal, QC, Canada, H3A 2R7, 2015). All simulations had GTP nucleotide and coordinating Mg2+ion bound. The system was embedded into a water box with 12 Å buffer, containing charge neutralizing Na+ ions. The protein was modelled with Amber force field ff19SB (Tian, C., et al, J. Chem. Theory Comput. 2020, 16, 528-552), water with TIP3P (Jorgensen et al., J. Chem. Phys 1983, 79, 926-935) and ions with JC parameters (Joung et al., J. Phys. Chem. B 2008, 112, 9020-9041. Each system was first equilibrated for 100 ns in the NPT ensemble before initiating 1 μs NPT production runs. Simulations were performed in quadruplicate, amounting to 4 us production time per KRAS mutant.

Analysis of the molecular dynamics trajectories was performed with CPPTRAJ (Roe et al., J. Chem. Theory Comput 2013, 9, 3084-3095). The per-residue root-mean-square fluctuation (RMSF) was calculated per production run with final results taken as an average across the four runs per mutant.

On analysis of the RMSF data, G13R and Q61R were found to display similar dynamics during the simulations, both having a mobile “switch-I” loop and more restrained “switch-II” region. It may thus be concluded that the G13R and Q61R mutants overlap dynamically and presumably adopt similar conformations.

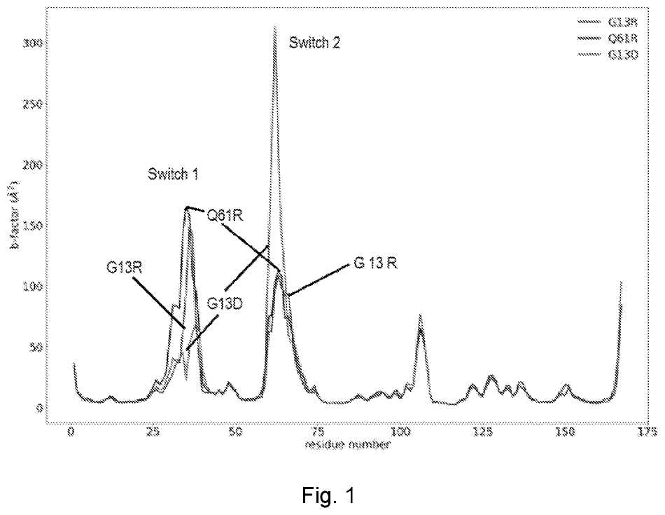

FIG. 1 shows the corresponding results and confirms that KRAS G13R protein dynamic overlaps KRAS Q 61R but not G13D.

### Example 3: Study on Treatment of Solid Tumors with a RAS Codon Q61 or G13R Mutation with a Combination of Naporafenib and Trametinib

The following clinical trial may be carried out: an open-label, non-randomized, Phase II study to assess the efficacy and safety of naporafenib in combination with trametinib in subjects with unresectable or metastatic melanoma with a codon Q61 or G13R NRAS mutation who have received prior anti-PD (L)-1 with or without anti-CTLA-4 or anti-LAG-3, and in subjects with advanced solid tumors with a RAS codon Q61 or G13R mutation who have progressed on available standard of care or for whom no standard treatment options are available. ORR, as primary endpoint, may be based on blinded independent review committee (BIRC) using Response Evaluation Criteria in Solid Tumors (RECIST) version 1.1. PFS, OS and DoR are secondary endpoints. Patients with NRAS mutant melanoma may be selected for enrollment into the clinical study via a single central diagnostic test. This investigational clinical trial assay is intended to be the test registered as the companion diagnostic.

Subjects may be allocated as follows:
Cohort 1: Adult (≥18 years) subjects with codon Q61 or G13R NRAS mutant unresectable or metastatic melanoma who progressed on treatment with PD-(L)-1 inhibitors alone or in combination CTLA4 or LAG 3 inhibitors.
Cohort 2: Adult (≥18 years) subjects with codon Q61 or G13R RAS mutant advanced metastatic solid tumors who have progressed on available standard of care or for whom no standard treatment options are available as determined by locally/regionally standard practice and the treating physician's discretion.

Subjects may receive naporafenib 200 mg orally twice daily and trametinib 1 mg orally once daily until progressive disease per RECIST 1.1, unacceptable toxicity or death, loss to follow-up, or withdrawal of consent.

This study design aims to further assess the novel combination of naporafenib plus trametinib that has shown encouraging activity and acceptable toxicity in a Phase Ib study CLXH254X2102 (see Example 1) in a population with a poor prognosis and high unmet medical need which is characterized by the mutations mentioned.

Clinical studies and internal unpublished data suggest limited activity of both trametinib and naporafenib when administered alone in patients with NRAS mutated melanoma (Falchook et al 2012; Lancet Oncol. 13 (8): 782-9). Single agent MEK inhibitor such as binimetinib from the NEMO study (Dummer et al 2017; Lancet Oncol. 19, 1315-27) also exhibited limited anti-tumor activity in NRAS mutant melanoma.

In view of the data and information herein, however, a combination of naporafenib and trametinib shows potential in the treatment of solid tumors (and also (especially unresectable) melanoma.

Mutations in NRAS activate intracellular a variety signaling through of pathways including RAS-RAF-MAPK pathway. It is also known that these mutations activate the MAP kinase pathway to the same extent as BRAF V600 mutations. Given RAF and MEK are vertically integrated in the same pathway, a combined inhibition of these two nodes can provide more effective inhibition as supported by preclinical and clinical data. This study evaluates the efficacy of naporafenib (RAF inhibitor) in combination with trametinib (MEK inhibitor) in subjects with codon Q61 or G13R NRAS mutant unresectable or metastatic melanoma who progressed on treatment with PD-(L)-1 inhibitors alone and/or in combination CTLA-4 or LAG 3 inhibitors, and especially in subjects with codon Q61 or G13R RAS mutant advanced metastatic solid tumors who progressed on available standard of care or for whom no standard treatment options are available.

The main subject population characteristics are:

### Key Inclusion Criteria

### Cohort 1:

Histologically confirmed unresectable stage IIIB/C/D or metastatic/stage IV melanoma using AJCC Edition 8.
Previously treated for unresectable or metastatic melanoma. Subjects must have received prior systemic therapy for unresectable or metastatic melanoma with anti-PD-(L)-1 agent with or without anti-CTLA-4 or anti-LAG (no more than 2 lines of anti-PD (L)-1 based regimen).
Confirmation of codon Q61 or G13R NRAS mutation in tumor tissue at a laboratory, even when local test result is available. All subjects must provide fresh or archival tumor sample from metastatic setting for NRAS mutation testing

### Cohort 2:

Histologically confirmed solid tumor of advanced disease (AJCC Edition 8) and no available standard treatment options as determined by locally/regionally standards of care and treating physician's discretion.
Confirmation of codon Q61 or G13R RAS (including HRAS, KRAS and NRAS) mutation in tumor tissue at a Novartis designated laboratory, even when local test result is available. All subjects must provide fresh tumor or archival sample from metastatic setting for RAS mutation testing.

### All Cohorts:

Age (male or female) 18 years or older at the time of the informed consent
ECOG performance status 0, 1 or 2.
The last dose of prior therapy must have been received more than four weeks before randomization
Must have at least one measurable lesion per RECIST v1.1.

### Key Exclusion Criteria

### Cohort 1:

Subjects with uveal or mucosal melanoma
Prior systemic therapy for unresectable or metastatic melanoma with any investigational agent, or with any agents other than anti-PD-(L)-1 monotherapy or anti-PD-(L)-1 in combination with anti-CTLA-4 or anti-LAG-3

### Cohort 2:

Subjects with NRAS melanoma mutation
Prior treatment with chemotherapy, immunotherapy, biologic therapy or chemoradiation with delayed toxicity within 4 weeks prior to enrollment. Prior treatment with investigational product(s) within 4 weeks or within 5 half-lives (whichever is longer) prior to enrollment

### All Subjects:

Prior systemic therapy of BRAF inhibitor and/or MEK inhibitor
Prior solid organ transplantation or stem cell transplantation
Radiation therapy ≤4 weeks prior to start of study treatment (Note: Palliative radiotherapy for bone lesions is allowed) or active CTCAE grade ≥2 radiotherapy-related toxicities ≤2 weeks prior to start of study treatment
Prior major surgery less than 14 days prior to enrollment. Any surgery-related AE(s) must have resolved prior to enrollment
Primary CNS tumors or presence of clinically active or unstable brain metastasis at time of screening; or symptomatic or untreated leptomeningeal or spinal cord compression. Note: Subjects with previously unstable brain lesions who have been definitively treated with stereotactic radiation therapy, surgery or gamma knife therapy are eligible.
   Subjects with brain lesions who are untreated (including newly discovered brain lesions during screening) or received intracranial radiation must have documented stable disease as assessed by two consecutive assessments ≥2 weeks apart and have not required high dose steroids for at least ≥4 weeks prior to randomization/enrollment.
History of another malignancy including hematologic malignancies
   NOTE: Subjects with another malignancy are eligible if they:
      are disease-free for 3 years, or
      have a history of completely resected non-melanoma skin cancer, and/or
      completely resected carcinoma in situ of any type
Clinically significant GI abnormalities that may alter absorption such as malabsorption syndrome or major resection of the stomach or bowels
History or current evidence/risk of retinal vein occlusion (RVO) or central serous retinopathy
Systemic chronic steroid therapy (≥10 mg/day prednisone or equivalent) or any immunosuppressive therapy 7 days prior to planned date for first dose of study treatment. Topical, inhaled, nasal and ophthalmic steroids are allowed.
Current use of prohibited medication(s) or requirement for prohibited medications during study
Cardiac disease or cardiac repolarization abnormality, including any of the following:
   Congestive heart failure requiring treatment (New York Heart Association Grade ≥2), LVEF <50% as determined by multiple gated acquisition (MUGA) scan or echocardiogram (ECHO), or uncontrolled hypertension defined by blood pressure ≥140 (systolic)/90 (diastolic) mmHg at rest (average of 3 consecutive readings) despite medical treatment.
   History of myocardial infarction, angina pectoris, coronary artery bypass graft within 6 months prior to starting study treatment
   History or presence of clinically significant cardiac arrhythmias (e.g., ventricular tachycardia), complete left bundle branch block, high-grade AV block (e.g., bifascicular block, Mobitz type II and third-degree AV block
   Long QT syndrome, family history of idiopathic sudden death or congenital long QT syndrome, or any of the following:
      Risk factors for Torsades de Pointe including uncorrected hypokalemia or hypomagnesemia, history of cardiac failure, or history of clinically significant/symptomatic bradycardia
      Concomitant medication(s) with a known risk to Torsades de Pointe that cannot be discontinued or replaced by safe alternative medication within 7 days prior to start of treatment
      Inability to determine the QTcF interval
Abnormal ECG as determined by the mean of a triplicate ECG and assessed by the central laboratory at screening:
   Resting heart rate <60 or >90 bpm
   QTcF at screening ≥450 msec (male) or ≥460 msec (female) (using Fridericia's correction).

### Study Treatments

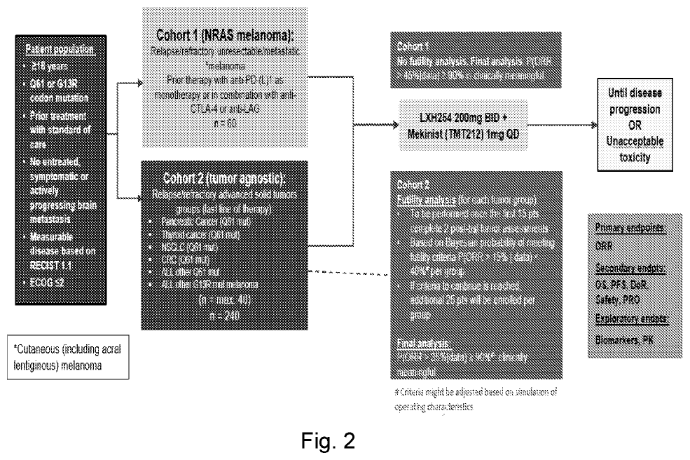

This study evaluates the combination of naporafenib, a Raf inhibitor with trametinib a MEK inhibitor in patients with unresectable or metastatic codon Q61 or G13R NRAS-mutated melanoma, and subjects with codon Q61 or G13R RAS mutant advanced metastatic solid tumors. Different RAS variants display distinct patterns of interactions with downstream effector molecules. Considerable combination activity for naporafenib and trametinib was seen in NRAS melanoma cell lines. The combination of naporafenib and trametinib was synergistic in all lines tested, with the strongest synergy observed in MM127 (NRASG13R), SK-MEL-2 (NRASQ61R) and MEL-JUSO (NRASQ61L). Combination of naporafenib and trametinib was superior to both agents alone in reducing phosphorylation of MEK1/2, ERK1/2, and RSK3. Additionally, the combination of low dose trametinib and naporafenib was equivalent or superior to a 10-fold higher single agent dose of naporafenib. In mouse xenografts (PTX) models, the combination of naporafenib with MEK inhibitor (trametinib achieved better control of tumor growth than each drug alone (3. FIG. 2 gives a representation of results of anti-tumor activity of naporafenib and trametinib across ten patients derived NRASmut melanoma tumor xenograft models in mice.

Clinical data further supports the pre-clinical findings. Trametinib has shown limited single agent activity in RAS-driven tumors (i.e. a Phase II randomized trial failed to demonstrate superiority against standard of care docetaxel in second line KRAS G12, G13C and G13D mutant NSCLC-Blumenschein et al 2015, Ann. Oncol. 26 (5), 894-901). A phase 1 first in human (FIH) dose escalation study evaluated trametinib single agent, in 97 patients with advanced melanoma, of which 7 had a NRAS mutation. In these 7 patients, trametinib monotherapy (continuous doses 2 mg, 2.5 mg, 3 mg; 2 week run-in with dose increase 1/2 mg, 0.5/2.5 mg, and 0.5/2.5 mg; 3 week on 1 week off 2 mg) showed limited activity, with no responses and 2 patients with stable disease (SD) (Falchook et al 2012; Lancet Oncol. 13 (8), 789-9). Similarly, the phase 1 FIH study CLXH254X2101 of naporafenib single agent in patients with solid tumors harboring MAPK alterations, enrolled 10 patients with NRAS mutated melanoma and showed no responses and 4 SDs across different doses (doses 400-1200 qd and 200-800 bid) (data cut of May 31, 2021). These studies suggest limited activity of both trametinib and naporafenib when administered alone, consistent with the pre-clinical studies results.

In contrast, the combination of 200 mg naporafenib and 1 mg trametinib was explored in Study CLXH254X2102 in patients with NRAS mutated advanced or metastatic melanoma. Sixteen patients were treated with the combination of naporafenib 200 mg and trametinib 1 mg. This combination led to a 43.8% confirmed overall response rate (ORR), with 3.76 months of median duration of response (DOR), 5.5 months of mPFS, 8.8 months of mOS, and a 12-month OS rate of 48.9% (see Table 1 in Example 1).

The above data supports the combination of naporafenib and trametinib in patients with NRAS mutant melanoma versus each agent alone.

Based on this data and that in Example 1, a particularly preferred dosing regimen useful in the methods of the present invention isnaporafenib 200 mg bid+trametinib 1 mg qd

### Endpoints and Estimates

ORR assessed by Blinded independent review committee (BIRC) per RECIST 1.1 Response Evaluation Criteria in Solid Tumors, version 1.1) is the primary endpoint, in order to avoid potential investigator bias.

ORR is chosen as a primary endpoint because it is a direct measure of drug anti-tumor activity which can be evaluated in single-arm trials (FDA Guidance for Industry, 2018; Clinical Trial Endpoints for the Approval of Cancer Drugs and Biologics (fda.gov). Source URL: https://www.fda.gov/media/71195/download).

PFS, OS and Duration of Response (DOR) are secondary endpoints. PFS is defined as the time from randomization until the earliest of documented disease progression by BIRC per RECIST 1.1 or death due to any cause. OS is defined as the time from randomization until death due to any cause.

DOR is also an important factor in the assessment of the trial outcome, and is defined for the subset of subjects who have a confirmed overall response by BIRC as the time from the first radiological evidence of confirmed overall response by BIRC until the earliest of documented disease progression by BIRC per RECIST 1.1 or death due to any cause.

The present invention provides a method of treatment of a solid tumor wherein a pharmaceutical combination of naporafenib and trametinib is administered to subjects with unresectable or metastatic Q61/G13R NRAS mutant melanoma that progressed on anti-PD-(L)-1 (alone or in combination with anti-CTLA-4 or anti-LAG-3) or RAS Q61/G13 mutant advanced metastatic solid tumors who have progressed on available standard of care or for whom no standard treatment options are available. The patients to be treated by the methods of the invention and the pharmaceutical combinations of the invention thus include patients with unresectable or metastatic Q61 or G13R NRAS mutant melanoma who have progressed on anti-PD-(L)-1 therapy (alone or in combination with anti-CTLA-4 or anti-LAG-3) or RAS Q61/G13 mutant advanced metastatic solid tumors who have progressed on available standard of care or for whom no standard treatment options are available.

Previous therapy for such patients includes the following.

In general, these patients are treated as metastatic melanoma in 2L/3L regardless of NRAS mutation after anti-PD-(L)-1 progressions with ipilimumab (ORR 13% Da Silva 2021; Lancet Oncol. 22 (6), 836-847), IL-2 (ORR 16%, Atkins et al 1999; J. Clin. Oncol. 17 (7), 2105-16), paclitaxel/carboplatin (ORR 26%, Rao et al 2006; Cancer 106 (2), 375-82), dacarbazine (ORR 7-20% Dummer et al 2017, loc. cit.; Serrone et al 2000; J. Exp. Clin. Cancer Res. 19 (1), 21-34) and temazolamide (ORR 13.5%, Rao et al 2006; loc. cit.). Approved alternative treatments for other solid tumor types are not specific for RAS isoform with the exception of KRAS G12C inhibitor (sotorasib) for NSCLC where ORR is 36% and a median DoR of 10 months.

### INCORPORATION BY REFERENCE

All clinical trials, publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations. Deletion of this paragraph caused by official or court objections to its wording is not meaning to diminish a protection scope including equivalency protection.

1. An agent or a probe capable of identifying a RAS codon Q61 and/or (especially) G13R mutation for (or in particular for use in) diagnosing a solid tumor, in particular other than melanoma, yet more preferably other than melanoma or other than rhabdomyosarcoma, for amenability to a combination treatment with a Raf inhibitor and a MEK inhibitor, said diagnosing comprising applying the agent to assay a sample of said solid tumor whether it harbors a RAS codon Q61or RAS G13R mutation as biomarker for said amenability.

2. A method of identifying a patient suffering from a solid tumor, in particular other than melanoma, yet more preferably other than melanoma or other than rhabdomyosarcoma, for amenability to a combined treatment with a Raf inhibitor and a MEK inhibitor, the method comprising:
(a) assaying a tumor sample obtained from the patient for the presence of a RAS codon Q61 or RAS G13R mutation with an agent according to the preceding paragraph; and
(b) selecting a patient whose tumor sample was positively assayed under (a) for the presence of said RAS codon Q61 or RAS G13R mutation as candidate for said combination treatment.

3. A method for selecting a treatment for a patient suffering from a solid tumor, the method including screening a tumor sample from the patient for a RAS codon Q61or RAS G13R mutation, wherein the presence of said mutation(s) in the tumor sample identifies the patient as one who may be expected to benefit from a treatment including a Raf inhibitor and a MEK inhibitor.

4. A method of treating a patient suffering from a solid tumor, the method comprising
(a) examining a tumor sample from the patient suffering from the tumor whether it harbors a RAS codon Q61 or RAS G13R mutation, and,
(b) where the tumor sample has been determined to harbor said mutation, administering in a combined treatment of therapeutically effective amounts a Raf inhibitor and a MEK inhibitor.

5. A method of treatment of a patient suffering from a solid tumor, in particular other than melanoma, in need of such treatment, comprising administering to said patient, where the solid tumor was identified to harbor a RAS codon Q61 or RAS G13R mutation, a combination of, in an effective amount, respectively, a RAF inhibitor and a MEK inhibitor.

6. A kit, comprising an agent for identifying a RAS codon Q61 or RAS G13R mutation and reagents for a method according to any one of the preceding paragraphs.

7. A Raf inhibitor and a MEK inhibitor for combined use in the treatment of a solid tumor, in particular other than melanoma and/or rhabdomyosarcoma harboring a NRAS Q61 mutation, in a patient where the tumor has been positively tested and identified to harbor a RAS codon Q61 or RAS G13R mutation.

8. A Raf inhibitor and a MEK inhibitor for combined use according to claim 7, where the Raf inhibitor is selected from the group consisting of encorafenib; sorafenib, vemurafenib, dabrafenib, GDC-0879, PLX-4720, PLX8394, belvarafenib, CCT3833/BAL3833, LY3009120, LSN3074753, lifirafenib, BGB659, RO5126766, AZ-628, MLN2480, BeiGene-283, RXDX-105, BAL3833, INU152, regorafenib, tovorafenib, Day 101, TAK580, MLN 2480, TAK632 and especially naporafenib (LXH254; Type II); or a pharmaceutically acceptable salt, hydrate or solvate thereof, respectively.

9. A Raf inhibitor and a MEK inhibitor for combined use according to any one of claim 7 or 8, where the Raf inhibitor is naporafenib, or a pharmaceutically acceptable salt, hydrate or solvate thereof.

10. A Raf inhibitor and a MEK inhibitor for combined use according to any one of claims 7 to 9, wherein the MEK inhibitor is selected from the group consisting of binimetinib, trametinib, selumetinib, cobimetinib, CI-1040, U0126-EtOH, PD198306, PD98059, BIX 02189, TAK-733, Honoliol, AZD8330, PD318088, BIX 02188, pimasertib, mirdametinib, refametinib, BI-847325, GDC0623, G-573, trametiglue, RO5126766 and pimisertib, or a pharmaceutically acceptable salt, hydrate or solvate thereof.

11. A Raf inhibitor and a MEK inhibitor for combined use according to any one of claims 7 to 10, wherein the MEK inhibitor is trametinib, or a pharmaceutically acceptable salt, hydrate or solvate thereof.

12. A Raf inhibitor and a MEK inhibitor for combined use according to any one of claims 7 to 11, where the solid tumor has been positively tested to harbor a G13R mutation.

13. The combined use of a Raf inhibitor and a MEK inhibitor, especially as defined in more detail herein, especially naporafenib and trametinib, or a pharmaceutically acceptable salt, hydrate or solvate thereof, respectively, in the treatment of a solid tumor in a patient, where the tumor has been positively tested to harbor a RAS codon Q61or RAS G13R mutation.

14. The use of a Raf inhibitor and a MEK inhibitor, especially as defined claims 8 and 10, respectively, especially naporafenib and trametinib, or a pharmaceutically acceptable salt, hydrate or solvate thereof, respectively, in the preparation of (especially combination) medicaments, e.g. kits for combined use or fixed combination formulations comprising both active ingredients, for combination treatment of a solid tumor in a patient, where the solid tumor has been determined to harbor a RAS codon Q61or RAS G13R mutation.

15. A composition comprising a Raf inhibitor and a MEK inhibitor, especially as defined in more detail in claim 10 and claim 12, respectively, especially naporafenib and trametinib, or a pharmaceutically acceptable salt, hydrate or solvate thereof, respectively, and a pharmaceutically acceptable carrier in the form of separate formulations or a kit or a fixed formulation for use in the treatment of a solid tumor selected from rhabdomyosarcoma identified to harbor a RAS codon G13R mutation, or a different solid tumor, especially other than melanoma, identified to harbor a RAS codon Q61or RAS G13R mutation; or a corresponding method of treatment comprising administering the Raf inhibitor and MEK inhibitor mentioned in a combined therapeutically active amount to a patient suffering from said rhabdomyosarcoma or other solid tumor in need of such treatment.
